(19) United States
(12) Patent Application Publication
(10) Pub. No.: US 2022/0409406 A1
(43) Pub. Date: Dec. 29, 2022

(54) FLEXIBLE INNER SOCKET FOR PROVIDING INNER CIRCUMFERENCE REDUCTION TO RIGID PROSTHETIC SOCKET

(71) Applicant: Medical Creations, Inc., Denver, CO (US)

(72) Inventors: Mark C. Joseph, Aspen, CO (US); Sean D. Smith, Denver, CO (US)

(21) Appl. No.: 17/901,375

(22) Filed: Sep. 1, 2022

Related U.S. Application Data
(63) Continuation-in-part of application No. 16/897,425, filed on Jun. 10, 2020.

(51) Int. Cl. A61F 2/78; A61F 2/60; A61F 2/80

(52) U.S. Cl. CPC A61F 2/7812; A61F 2/60; A61F 2/80; A61F 2002/5052

(57) ABSTRACT

A flexible prosthetic inner socket that resides inside of a rigid prosthetic socket that is fabricated by forming a sized, injection molded, flexible inner pre-socket. The flexible inner pre-socket includes a body formed with an opening and an enclosed end. The Pre-socket can be heated and stretched over a prosthetic residual limb model and then a prosthetic rigid socket can be formed over it. It can also be heated and vacuum-formed inside of an existing hard prosthetic socket in order to provide global volume reduction intended to make the rigid prosthetic socket fit tighter onto the residual limb. The opening of the flexible inner socket may be trimmed after the formation to fit contours of an opening of the rigid prosthetic socket.

### CROSS REFERENCE TO RELATED APPLICATIONS

The present application is continuation-in-part of U.S. application Ser. No. 16/897,425 filed on Jun. 10, 2020, which is fully incorporated herein by reference as if fully set forth herein.

### BACKGROUND OF THE INVENTION

### Field of the Disclosure

The present disclosure generally relates to a flexible inner socket that can be placed within a prosthetic socket, method of using and making the same and more particularly to flexible inner socket for providing at least a partial inner circumference reduction of the prosthetic socket.

### SUMMARY OF THE INVENTION

Accordingly, the invention is directed towards a flexible inner socket for providing inner circumference reduction to prosthetic socket.

An advantage of the invention is to provide a process that is reproducible and faster than related art processes.

Another advantage of the invention is to provide accurate and precise global reduction of the fit of a prosthetic socket that is based on the thickness of the flexible inner socket instead of the common laborious practice in the art of hand scraping, grinding, and sanding and the like.

Yet another advantage of the invention is to provide one or more flexible inner sockets in varied thicknesses that can precisely reduce the global reduction of the fit as the user's limb changes size during the day.

Additional features and advantages of the invention will be set forth in the description which follows, and in part will be apparent from the description, or may be learned by practice of the invention. The objectives and other advantages of the invention will be realized and attained by the structure particularly pointed out in the written description and claims hereof as well as the appended drawings.

To achieve these and other advantages and in accordance with the purpose of the present invention, as embodied and broadly described, relate to a flexible inner socket design and process of fabricating a flexible inner socket. The flexible inner socket is formed on an inner surface of a prosthetic socket that has previously been formed to fit a residual limb of a user. The prosthetic socket was formed either directly over the residual limb of the user or over a model of the residual limb that has a shape matching the anatomical shape of at least a portion of the residual limb. Accordingly, the inner surface of the prosthetic socket has a contour and dimensions that match the anatomical shape of at least a portion of the residual limb. Zero or insufficient global reduction has been applied to the rigid prosthetic socket, or the residual limb has shrunk to the extent that the original global reduction is no longer effective. The flexible inner socket, when attached to the inner surface of the rigid prosthetic socket, adds additional thickness to the prosthetic socket so that the resulting device (i.e., the prosthetic socket plus the flexible inner socket) has a reduced inner circumference. This is referred to as global interior circumference reduction or global reduction of the prosthetic socket. The global reduction can be precisely controlled based on the thickness of the flexible inner socket.

In another aspect of the invention, an embodiment is directed towards a flexible inner socket made using a preformed flexible inner socket (also referred to as “flexible inner pre-socket”). The flexible inner pre-socket is formed from a material, e.g., polymer, thermoplastic material, and the like, using techniques such as injection molding, rotational molding, three-dimensional (3D) printing, blow molding, combinations of the same and the like. The flexible inner pre-socket has an opening (e.g., the opening through which the residual limb will ultimately be inserted) and an enclosed end (e.g., where the residual limb ultimately rests). The enclosed end is opposite the opening. The flexible inner pre-socket has an outer circumference that is smaller than the inner circumference of the rigid prosthetic socket. In one embodiment, the flexible inner pre-socket is heated and then molded onto the inner surface of the rigid prosthetic socket to form the flexible inner socket. In another aspect of the invention, an embodiment is directed towards a method of forming a flexible inner socket including providing a rigid prosthetic socket configured to fit at least a portion of a residual limb of a user. The rigid prosthetic socket comprises a first end, a second end, an inner circumference, the first end having an opening, the second end being substantially closed, and an inner surface having contours that substantially mimic contours of the residual limb. The rigid prosthetic socket has not been globally reduced in dimension. The method also includes providing a flexible inner pre-socket comprising a thermoformable material. The flexible inner pre-socket includes a first end, a second end, a thickness, an inner circumference, and an outer circumference, the first end having an opening, and the second end being substantially closed. The method further includes heating the thermoformable material of the flexible inner pre-socket to a temperature so that the flexible inner pre-socket becomes a formable flexible inner pre-socket and arranging at least a portion of the formable flexible inner pre-socket into the rigid prosthetic socket, thereby forming a flexible inner socket. Next the heated flexible inner pre-socket is molded onto the inner surface of the rigid prosthetic socket such that the outer circumference of the formable flexible pre-socket substantially follows the contours of the inner surface of the rigid prosthetic socket, thereby reducing the inner circumference of the rigid prosthetic socket in a predetermined manner based on the thickness of the flexible inner pre-socket to provide a globally reduced fit of the rigid prosthetic socket.

Yet another aspect of invention an embodiment is directed towards a method of forming a flexible inner socket including providing a rigid prosthetic socket configured to fit at least a portion of a residual limb of a user. The rigid prosthetic socket includes a first end, a second end, an inner circumference, the first end having an opening, the second end being substantially closed, and an inner surface having contours that substantially mimic contours of the residual limb, and the rigid prosthetic socket has not been globally reduced in dimension. The method also includes providing a flexible inner pre-socket and heating the flexible inner pre-socket to a temperature so that the flexible inner pre-socket becomes a moldable. The heated flexible inner pre-socket is then arranged into the rigid prosthetic socket. Next, heated flexible inner pre-socket is molded onto the inner surface of the rigid prosthetic socket such that the outer circumference of the formable flexible inner pre-socket substantially follows the contours of the inner surface of the rigid prosthetic socket.

In still another aspect of the invention, an embodiment is directed towards a method of forming a flexible inner socket. The method includes providing a prosthetic socket having an inner surface with contours that substantially mimic contours of the residual limb and providing a flexible inner pre-socket. The flexible inner pre-socket is heated with a heat source to a temperature where the pre-socket becomes a moldable and at least a portion of the flexible inner pre-socket is arranged into the prosthetic socket. Next, the heated flexible inner pre-socket is molded onto the inner surface of the rigid prosthetic socket such that an outer circumference of the flexible inner pre-socket substantially follows one or more of the contours of the inner surface of the rigid prosthetic socket.

Yet still another aspect of the invention, an embodiment is directed towards a prosthetic system for an end user. The system includes a rigid prosthetic socket configured to fit at least a portion of a residual limb of a user. The rigid prosthetic socket includes a first end, a second end, an inner circumference, the first end having an opening, the second end being closed, and an inner surface having contours that substantially mimic contours of the residual limb and the rigid prosthetic socket has not been globally reduced in dimension. The system further includes a molded flexible inner pre-socket comprising a thermoformable material, wherein the flexible inner pre-socket comprises a first end, a second end, a thickness, an inner circumference, and an outer circumference, the first end having an opening, and the second end being closed. The molded flexible inner pre-socket substantially follows the contours of the inner surface of the rigid prosthetic socket, thereby reducing the inner circumference of the rigid prosthetic socket in a predetermined manner based on the thickness of the flexible inner pre-socket to provide a globally reduced fit of the rigid prosthetic socket.

Yet still another aspect of the invention, an embodiment is directed towards a prosthetic system for an end user. The air inlet assembly for use with a flexible inner pre-socket includes a sealing mechanism, a lid having a first side, a second side, a valve and a channel region around a circumference of the lid, the channel region configured to receive the sealing mechanism and at least a portion of the flexible inner pre-socket. The air inlet assembly further includes a hose configured to be releasably connected to the valve and the lid is configured to cover an opening on the flexible inner pre-socket and the sealing mechanism is configured to seal the lid to the flexible inner pre-socket. Unlike conventional methods of fabrication where the flexible inner socket is formed on a plaster cast of the limb and where the rigid prosthetic socket is formed over the flexible inner socket, the flexible inner socket and method of fabricating described here include actually forming the flexible inner socket inside the rigid prosthetic socket, where the prosthetic socket was previously formed to fit the user's residual limb, without global reduction being applied. Also, the flexible inner socket is formed from a preformed socket that can be made by using techniques such as injection molding, rotational molding, 3D printing, blow molding, combinations of the same and the like, as opposed to hand crafting in the conventional methods. Thus, the inner circumference reduction provided by the flexible inner socket here can be more accurate and precise. The techniques can be used to manufacture flexible inner pre-sockets of various shapes and sizes to fit rigid prosthetic sockets of various shapes and sizes.

This Summary section is neither intended to be, nor should be, construed as being representative of the full extent and scope of the present disclosure. Additional benefits, features and embodiments of the present disclosure are set forth in the attached figures and in the description hereinbelow, and as described by the claims. Accordingly, it should be understood that this Summary section may not contain all of the aspects and embodiments claimed herein.

Additionally, the disclosure herein is not meant to be limiting or restrictive in any manner. Moreover, the present disclosure is intended to provide an understanding to those of ordinary skill in the art of one or more representative embodiments supporting the claims. Thus, it is important that the claims be regarded as having a scope including constructions of various features of the present disclosure insofar as they do not depart from the scope of the methods and apparatuses consistent with the present disclosure (including the originally filed claims). Moreover, the present disclosure is intended to encompass and include obvious improvements and modifications of the present disclosure.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

In the drawings:

FIG. 1A is a perspective view of a flexible inner socket, in accordance with an embodiment of the invention;

FIG. 1B illustrates the flexible inner socket of FIG. 1A arranged in a prosthetic socket of FIG. 1B, in accordance with the embodiment;

FIG. 1C illustrates a cross-sectional view of a residual limb of a user at least partially arranged in the flexible inner socket and the prosthetic socket of FIGS. 1A-1B, in accordance with the embodiment;

FIG. 2A is a perspective view of a flexible inner pre-socket, in accordance with another embodiment;

FIG. 2B is a cross-sectional view of the flexible inner pre-socket along, in accordance with another embodiment;

FIG. 2C is a top view of the flexible inner pre-socket, in accordance with another embodiment;

FIG. 2D is a bottom view of the flexible inner pre-socket, in accordance with another embodiment;

FIG. 3 illustrates a group of flexible inner pre-socket having different shapes and dimensions, in accordance with another embodiment;

FIG. 4 illustrates cross-sectional view of two flexible inner pre-sockets, each of which has a uniform thickness, in accordance with another embodiment;

FIG. 5 illustrates flexible inner pre-sockets having non-uniform thicknesses, in accordance with another embodiment;

FIG. 6 illustrates an air inlet assembly used for molding a flexible inner pre-socket, in accordance with another embodiment;

FIGS. 7A-7D illustrate a process of molding a flexible inner pre-socket onto an inner surface of a rigid prosthetic socket by using the air inlet assembly, in accordance with another embodiment;

FIG. 8 illustrates wraps used for constraining a flexible inner pre-socket during molding of the flexible inner pre-socket, in accordance with another embodiment;

FIG. 9 illustrates a process of molding a flexible inner pre-socket socket onto an inner surface of a rigid prosthetic socket by using an air bag, in accordance with another embodiment;

FIG. 10A is a perspective view of a system of molding a flexible inner pre-socket onto an inner surface of a rigid prosthetic socket by vacuum, in accordance with another embodiment;

FIG. 10B is a cross-sectional view of the system, in accordance with another embodiment;

FIG. 11 illustrates another system of molding a flexible inner pre-socket onto an inner surface of a rigid prosthetic socket by vacuum, in accordance with another embodiment;

FIGS. 12A-12B illustrate a process of trimming a flexible inner socket 1200, in accordance with another embodiment;

FIG. 13 illustrates a flow chart of a process for fabricating a flexible inner socket, in accordance with another embodiment;

FIG. 14A illustrates a mold configured to mimic a residual limb of a patient;

FIG. 14B illustrates a process of molding a flexible inner pre-socket onto a mold of FIG. 14A;

FIG. 14C illustrates a molded flexible inner pre-socket on a mold of FIG. 14A;

FIG. 14D illustrates a process of forming a prosthetic socket onto a flexible inner socket in a prosthetic socket according to another embodiment;

FIG. 14E illustrates a flexible inner socket and a prosthetic socket according to another embodiment;

FIG. 14F illustrates a process of trimming a flexible inner socket in a prosthetic socket according to another embodiment;

FIG. 14G illustrates a trimmed flexible inner socket arranged in a trimmed prosthetic socket according to another embodiment;

FIG. 15A illustrates a process of molding a flexible inner pre-socket onto a residual limb of a patient according to another embodiment;

FIG. 15B illustrates a process of molding a prosthetic socket onto a flexible inner socket on a residual limb of a patient according to another embodiment;

FIG. 16A illustrates an assembly used for molding a flexible inner pre-socket, in accordance with another embodiment;

FIG. 16B illustrates a partial cross-sectional view of a portion of FIG. 16A;

FIG. 16C illustrates a partial cross-sectional view of a portion of FIG. 16A on a portion of the flexible inner pre-socket; and

FIG. 16D illustrates a partial cross-sectional view of a portion of FIG. 16A on a portion of the flexible inner pre-socket.

### DETAILED DESCRIPTION

In the following description of embodiments, numerous specific details are set forth in order to provide more thorough understanding. However, note that the embodiments may be practiced without one or more of these specific details. In other instances, weft-known features have not been described in detail to avoid unnecessarily complicating the description.

Embodiments are described herein with reference to the figures where like reference numbers indicate identical or functionally similar elements. Also, in the figures, the left most digits of each reference number correspond to the figure in which the reference number is first used.

One embodiment relates to forming a flexible inner socket including the steps of providing a prosthetic socket configured to fit at least a portion of a residual limb of a user. The prosthetic socket includes a first end, a second end, an inner circumference, and an inner surface having contours that substantially match contours of the residual limb, wherein the prosthetic socket has not been globally reduced in dimension, was insufficiently globally reduced, or changes in the patient's residual limb have resulted in the global reduction to be insufficient. The process also includes forming a flexible inner pre-socket by using a thermoformable material, the flexible inner pre-socket comprising a first end, a second end, a thickness, an inner circumference, and an outer circumference, the first end having an opening, and the second end being closed. The process further includes heating the thermoformable material of the flexible inner pre-socket to a temperature so that the flexible inner pre-socket becomes a formable flexible inner pre-socket arranging the formable flexible inner pre-socket into the prosthetic socket. The process also includes molding the formable flexible inner pre-socket onto the inner surface of the prosthetic socket such that the outer circumference of the formable flexible inner pre-socket substantially follows the contours of the inner surface of the prosthetic socket, thereby reducing the inner circumference of the prosthetic socket in a predetermined manner based on the thickness of the flexible inner pre-socket to provide a globally reduced fit of the prosthetic socket.

The term flexible inner pre-socket and pre-socket are used interchangeably herein.

In one embodiment, a flexible inner pre-socket or pre-socket means a thermoplastic or thermoformable material that when heated, becomes pliable and stretchable so as to assume a new shape when formed and thereby holding that shape when cooled. In one embodiment, the heating temperature is in a range from about 170° F. to about 350° F. and the temperature when cooled is in a range from about 125° F. or below. In a preferred embodiment, the heating temperature is in a range from about 225° F. to about 280° F. (and when cooled about 150° F. or below). In a most preferred embodiment, the heating temperature 300° F. to about 315° F.

In one embodiment, the flexible inner pre-socket may have an open end and a substantially closed end. The substantially closed end may have a channel that extends from an inside portion to an outside portion through a thickness of the flexible inner pre-socket.

In one embodiment, the flexible inner pre-socket is heated, e.g., to a temperature in a range from about 190° F. to about 285° F. or greater to become pliable, and molded onto the inner surface of the rigid prosthetic socket. In a preferred embodiment, the flexible inner pre-socket is placed heated, to a temperature in a range from about 300 IF and 315° F. The flexible inner pre-socket can be heated with a heat source, e.g., an infrared heater, convection oven, silicone pad heater, halogen tube heater or other common heating devices.

Globally reduced means a reduction in the inner circumference of a lower leg prosthetic socket that is applied over the majority of the interior so that a user's residual limb soft-tissue is compressed in a conical fashion when worn so as to properly bear weight away from the amputated bone end. Global reduction is typically applied by hand filing, scraping and sanding of the plaster model which is an imprecise method. Global reduction is a common industry term well known and the typical reduction is about 4%.

In one embodiment, the thickness of the flexible inner pre-socket is determined based on the inner circumference of the prosthetic socket so as to achieve a desired global reduction according to Formula 1:

T=C/(2·π)·X/100 (Formula 1)

The units and variables are as follows: T is thickness of the flexible inner pre-socket [mm], C is inner circumference of the rigid prosthetic socket that has not been globally reduced in dimension [mm], and X is global reduction %.

In one embodiment, the flexible inner pre-socket becomes the final flexible inner socket after it is molded as described herein. In one embodiment, the flexible inner pre-socket can be molded directly onto a mold of a residual limb of a patient, an inner surface of a rigid prosthetic socket, and/or directly onto a residual limb of a patient.

In one embodiment, the thickness of the flexible inner socket can be controlled in the fabrication process (such as a manufacturing process), e.g., by controlling the thickness of the preformed flexible inner socket from which the flexible inner socket is formed. The preformed flexible inner socket may have a uniform thickness, or different portions of the preformed flexible inner socket can have different thicknesses. Accordingly, compression of the tissue of the user's residual limb can be precisely and selectively controlled to achieve proper distribution of the weight of the user and comfortable fit of the prosthetic socket.

In one embodiment, the flexible inner pre-socket can be formed with blow molding, injection material or other techniques. In one embodiment, the flexible inner pre-socket or socket is formed from a thermoformable material. The thermoformable material includes one or more of a thermoplastic polyurethane (TPU) material, a thermoplastic polyurethane foam material, a thermoplastic vulcanizate (TPV) material, thermoplastic elastomer (TPE) materials, a thermoset material, a rubber material, an ultra-low density polyethylene (ULDPE) material, an ethylene vinyl acetate (EVA) material, a styrene material and blends of the same.

In one embodiment, additional additives may be utilized and added to the thermoformable material of the pre-socket. The additives may include slip agent additives, e.g., PTFE, silicone, and the like, configured to allow the residual to slide into the flexible inner socket easier. Additional additives may also include antimicrobial and antibacterial additives and the like.

In one embodiment, the thermoformable material can include a closed cell foam material, a non-compressible material, or a compressible material. In one embodiment, the thermoformable e material has one or more of the following physical properties: a Shore A-type durometer in a range from about 50 to about 90 or Shore D-type durometer in a range from about 0 to 50, a density in a range from about 0.8 to about 1.2 g/cm³, flexural Modulus in a range of about 20 to about 35 MPa, a Tensile Modulus in a range of about 3 to about 10 MPa, a tensile Strength in a range of about 4 to about 10 MPA, a tear strength of about 30 to about 50 kN/m, an elongation at break in a range from about 300% to about 800%, and a forming point temperature in a range from about of about 170 to about 350° F.

In one embodiment, the flexible inner pre-socket is formed with an opening and with an enclosed end that is opposite to the opening. The flexible inner pre-socket has a circumference determined based on an inner circumference of a prosthetic socket, e.g., the circumference of the flexible inner pre-socket can be smaller than the inner circumference of the prosthetic socket, e.g., smaller by up to 15%, so that the flexible inner pre-socket can be arranged on inside the prosthetic socket. The prosthetic socket has previously been formed to fit a residual limb of a user with techniques described herein or conventional techniques. No global reduction has been applied or needed to the prosthetic socket.

In one embodiment, the flexible inner socket is formed after a flexible inner pre-socket is arranged inside the prosthetic socket. The flexible inner pre-socket is heated, so that is pliable and pressed onto the inner surface of the prosthetic socket such that the outer circumference of the heated flexible inner pre-socket substantially follows the contours of the inner surface of the prosthetic socket, thereby reducing the inner circumference of the prosthetic socket in a predetermined manner based on the thickness of the flexible inner pre-socket to provide a globally reduced fit of the prosthetic socket. After the flexible inner pre-socket is heated to a formable temperature it can be pressed into the prosthetic socket with an assistance of a pressured device, e.g., an airbag can be utilized to press against the inner surface of the heated preformed socket so that it molds against the inner surface of the prosthetic socket.

In one embodiment, a method of forming a flexible inner socket, by providing a prosthetic socket configured to fit at least a portion of a residual limb of a user, the prosthetic socket comprises a first end, a second end, an inner circumference, the first end having an opening, the second end being closed, and an inner surface having contours that substantially mimic contours of the residual limb, wherein the prosthetic socket has not been globally reduced in dimension. Next, providing a flexible inner pre-socket including a thermoformable material, the flexible inner pre-socket including a first end, a second end, a thickness, an inner circumference, and an outer circumference, the first end having an opening, and the second end being closed. The method further includes heating the flexible inner pre-socket to a temperature so that the flexible inner pre-socket becomes a formable flexible inner pre-socket and arranging at least a portion of the formable flexible inner pre-socket into the prosthetic socket. The method further includes molding the formable flexible inner pre-socket onto the inner surface of the prosthetic socket such that the outer circumference of the formable flexible inner pre-socket substantially follows the contours of the inner surface of the prosthetic socket, thereby reducing the inner circumference of the prosthetic socket in a predetermined manner based on the thickness of the flexible inner pre-socket to provide a globally reduced fit of the prosthetic socket.

Optionally and/or alternatively, the method further includes providing an air inlet assembly including a lid, a sealing mechanism, a valve in communication with the lid and a hose in communication with the valve. The lid is configured to cover the opening on the flexible inner pre-socket. The sealing mechanism is used to seal the lid to the flexible inner pre-socket and the entire assembly is arranged into at least a portion a prosthetic socket. The flexible inner pre-socket is inflated with the air inlet assembly to a pressure configured to conformally arrange at least a portion of an outer surface of the formable flexible inner pre-socket against an inner surface of the prosthetic socket.

In one embodiment, the flexible inner socket can be attached to the inner surface of the rigid prosthetic socket such that it holds the residual limb when a user wears the prosthetic socket. The flexible inner socket can be designed to be removable or non-removable from the prosthetic socket.

In one embodiment, the thickness of the flexible inner socket is predetermined and configured to provide a globally reduced fit of the prosthetic socket. In one embodiment, the inner circumference reduction of the prosthetic socket allows tissue of the residual limb of the user to be compressed against the material of the flexible inner socket. The compressing of the tissue in this manner more evenly distributes the weight born by the residual limb over the entire residual limb. The compressed tissue bears more weight over a larger surface area instead of bearing much of the weight at various points on the tissue that correspond to the bone ends and prominences of the residual limb. This allows the prosthetic socket to have a tight and comfortable fit.

In one embodiment, a rigid prosthetic socket is sized and fitted to the user's residual limb by various conventional techniques including making a plaster cast and filling the cast with plaster to make a model which the socket is subsequently made from. Optionally, a heat formable material can be direct formed to the plaster model or directly to the user's residual limb. The prosthetic socket would require a global reduction, e.g., about 4%, so that it fits tightly to the residual limb to properly fit and bear weight. In this embodiment, aspects of the invention and methods are configured to omit the global reduction step which is time consuming, requires considerable experience and technique, and often results in an imprecise shape and fit. In this case the socket is fitted precisely to the residual limb without any global reduction.

After the rigid prosthetic socket, which has not been reduced, was insufficiently globally reduced, or, as a result of residual limb volume reduction, provides insufficient global reduction the method of using and making a flexible inner socket is applicable. In this method, a global reduction is achieved by heat forming a flexible inner pre-socket precisely to an inside surface of the rigid socket. The flexible inner pre-socket is made from a material with properties described herein. In one embodiment, the flexible inner pre-socket becomes softened and formable at about 300° F.

In one embodiment, the flexible inner pre-socket is premade by injection molding or blow molding in economical repeatable quantities to a shape that fits inside the rigid prosthetic socket. The premade flexible inner pre-sockets can be made in different sizes so that the proper size is selected that fits closely inside the rigid prosthetic socket yet is taller so it extends out the proximal end. Also, they can be made in varied thicknesses as well so that the desired amount of global reduction is achieved. In one embodiment, the thickness of the pre-socket is about 2 mm thick and can fit a person with a medium size residual limb, measuring about 32 cm to about 34 cm at the distal end. The thickness after molding as described herein is configured to result in a global reduction, e.g., about 4%, Of course, other thickness can be utilized, e.g, according to Formula 1. The flexible inner pre-socket is molded into a flexible inner socket as described herein. Next it can be marked for trimming so that it extends about 2 cm above the edge of the rigid prosthetic socket. It is removed, trimmed and the edge is buffed round and smooth with a rotating buffing tool.

Optionally and/or alternatively, after the flexible inner socket is fitted or molded back inside the rigid prosthetic socket, the extending edge can be heated, e.g., about 250 with a heat source, e.g., a heat gun, and it can be shaped, e.g., flared by gloved hands to precisely fit the needs of the residual limb. Depending on the suspension system desired to retain the socket onto the limb, a small hole can be cut in the distal end to allow for suction or vacuum air to pass through. Or, a larger hold can be cut to allow for a pin lock system to pass through.

In one embodiment, the system includes a rigid prosthetic socket and flexible inner socket, which can be tested for fit by the user. If the fit needs adjustment, heat can be applied with a heat source to the flexible inner socket to reshape it. In a preferred embodiment, the rigid prosthetic socket is also heat formable and adjusted as described with referent to with reference to U.S. Pat. Nos. 15/914,480 and 16/516,199, both of which are hereby incorporated by reference as if fully set forth herein. In such a case, both the flexible inner socket and rigid prosthetic can heated and adjusted in shape for a truly customizable fit that can be done at any time. The result is a precisely globally reduced socket that fits to compress the limb properly to bear weight and stay in place which is far superior to the typical hand reduction done to the plaster limb model by the practitioner. A typical problem for amputees is that their limb can shrink or grow in circumference at different times through the day, week, month, and/or year. This typically occurs as the day passes and use reduces fluid in the limb but it can happen at any time, Additional flexible inner sockets can be made in varied thickness that the user can insert into the rigid prosthetic socket to increase or reduce global reduction at any time of the users. They can replace the original flexible inner socket or be layered to change the fit. These additional flexible inner sockets can be made in the same fashion described herein.

In one embodiment, a method includes forming a flexible inner socket with the use of a cast of the residual limb filled with plaster to create a positive model of the limb. The positive model is globally reduced using traditional techniques. A flexible inner pre-socket is formed over the globally reduced model, thereby creating a flexible inner socket with an inner surface that matches or mimics the surface and contours of the positive model that has been globally reduced. The flexible inner pre-socket thickness may be any thickness described herein. The flexible inner socket may be formed with hand pressure or with the aid of vacuum pressure or positive pressure, e.g., with apparatus and techniques described herein. A rigid prosthetic socket is then fabricated over the flexible inner socket still on the mold so that the inner surface of the rigid socket matches the surface and contours of the outer surface of the flexible inner socket. Use of the flexible inner pre-socket provides global reduction to the limb and/or improves edge pressures.

In one embodiment, a method is directed towards forming a flexible inner socket directly onto a residual limb of a patient. A flexible inner pre-socket is formed over the residual limb, thereby creating a flexible inner socket with an inner surface that matches or mimics the surface and contours of the residual limb. The flexible inner pre-socket thickness may be any thickness described herein. The flexible inner pre-socket may be formed by compressing the heated flexible inner pre-socket onto the residual with hand pressure or with the aid of vacuum pressure or positive pressure, e.g., with apparatus and techniques described herein. The flexible inner socket is allowed to cool to a temperature where it is no longer formable. A rigid prosthetic socket is then fabricated over the formed flexible inner socket still on the patient so that the inner surface of the rigid socket matches the surface and contours of the outer surface of the flexible inner socket.

Reference will now be made in detail to an embodiment of the present invention, example of which is illustrated in the accompanying drawings.

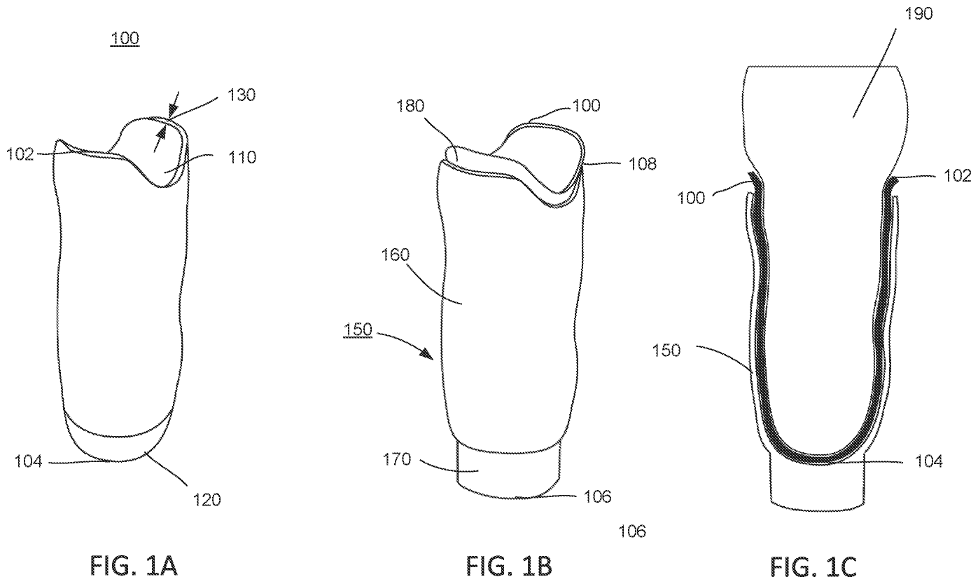

FIG. 1A is a perspective view of a flexible inner socket, in accordance with an embodiment of the invention. FIG. 1B illustrates the flexible inner socket of FIG. 1A arranged in a prosthetic socket of FIG. 1B, in accordance with the embodiment. FIG. 1C illustrates a cross-sectional view of a residual limb of a user at least partially arranged in the flexible inner socket and the prosthetic socket of FIGS. 1A-1B, in accordance with the embodiment;

Referring to FIGS. 1A-1C, a flexible inner socket formed as described herein is generally depicted with reference to 100 and a prosthetic socket is generally depicted with reference to 150. In a preferred embodiment, the prosthetic socket is rigid having a hardness of greater quantified as a Shore A-type durometer in a range from about 50 to about 90.

The flexible inner socket 100 has a first end 102, a second end 104, and the first end 102 has an opening 110 configured and dimensioned to receive at least a portion of a residual limb 190 of a user. The second end 104 is closed end 120 in a rounded type orientation. Optionally and/or alternatively, the second end has a hole or channel that extends through the thickness 130 of the flexible inner socket 100. A cavity or partial channel is formed extending from the open first end 102 to the closed second end 104. The cavity or channel is configured to enclose at least a portion of the residual limb 190. The volume of the cavity is greater than the volume of the residual limb. The flexible inner socket has a thickness 130. In a preferred embodiment the thickness is in a range from about 1 mm to about 8 mm. The thickness may be constant or variable about the x-axis or y-axis of the flexible inner socket. In one embodiment, the prosthetic socket 150 can be formed and is described with reference to U.S. Pat. Nos. 15/914,480 and 16/516,199, both of which are hereby incorporated by reference as if fully set forth herein. In yet another embodiment, the prosthetic socket is can be any conventional prosthetic socket as known it the related art.

The prosthetic socket 150 has a first end 108, a second end 106, and the first end 108 has an opening 180 configured and dimensioned to receive at least a portion of a residual limb 190 of a user and the flexible inner socket 100. The second end 106 is closed end 170. A cavity or partial channel is formed extending from the open first end 108 to the closed second end 106. The cavity or channel is configured to enclose at least a portion of the residual limb 190. The volume of the cavity is greater than the volume of the residual limb.

In one embodiment, a user can position the flexible inner socket 100 on at least a portion of the residual limb 190. Next, the user can position at least a portion of their residual into the prosthetic socket 150 by inserting the limb through the opening 180 of the first end. Moreover, the user can position their limb with the flexible inner socket 100 to the second end 106 until the base of the residual limb 190 rests on an inner surface of the enclosed end 106.

Referring to FIG. 1C, the residual limb 190 is shown arranged in the prosthetic socket 150. For example, a lower calf portion of a residual limb is inside the prosthetic socket 150 and inside the flexible inner socket 100. The prosthetic socket 150 attaches to a prosthetic mechanical lower leg to allow a person with a below or above the knee amputation to walk using the socket plus prosthesis (not shown).

The prosthetic socket 150 includes a cup portion 160 and a base portion 170. The cup 160 is configured in a shape of a hollow deep or elongated cup that is generally substantially cylindrical in shape and has an outer surface and an inner surface. The inner surface of the cup 160 is the inner surface of the prosthetic socket 150. The circumference of the inner surface is the inner circumference of the rigid prosthetic socket 150. The cup 160 is formed with an opening 180 via which the residual limb 190 can be inserted into the cup 160.

In this embodiment, the cup 160 is shapeable after being heated at a shaping temperature. The shaping temperature of the prosthetic socket can be in the range of about 150° F. to about 302° F. and any sub-range within. The shaping time can be in the range of about five minutes to about fifteen minutes, or any sub-range within. In one embodiment, the cup 160 has a pliability above a threshold pliability for a shaping time after being heated at the shaping temperature. The shaping time can be in the range of five minutes to fifteen minutes, or any sub-range within. During the shaping time, the cup 160 can be stretched circumferentially over the residual limb or a plaster model of the residual limb so that the cup 160 is shaped to fit the residual limb. No global reduction is applied to the cup 160 during the shaping. In one embodiment, the cup or prosthesis and its shaping is described with reference to U.S. patent Ser. No. 15/914,480 and 16/516,199 each of which is incorporated by reference as if fully set forth herein.

The base 170 joins the cup 160 to the prosthesis or is integral with the base unit. In one embodiment, the prosthesis can be a conventional prosthesis as known in the art or the prosthesis described in U.S. patent Ser. No. 15/914,480 and 16/516,199 each of which is incorporated by reference as if fully set forth herein. The base can have a pliability that is lower than the pliability of the cup 160 at the shaping temperature and/or at room temperature. Optionally and/or alternatively, the base 170 and the cup 160 are constructed from the same material. The base 170 is not heated or is heated at a lower temperature than the shaping temperature when the cup 160 is heated. Optionally and/or alternatively, the base 170 is made from a different material than the cup 160.

The prosthetic socket 150 is configured to have an inner dimension to fit at least a portion of the residual limb 190. For example, the prosthetic socket 150 may have been formed directly over the residual limb 190 or over a model of the residual limb 190 without global reduction being applied. The inner surface of the rigid prosthetic socket 150 can have a contour and dimension that match, e.g., the same as or substantially similar to, the anatomical shape and dimension of the residual limb 190.

In one embodiment, after the prosthetic socket 150 has been formed, it can used to fabricate the flexible inner socket 100. For example, a preformed flexible inner pre-socket having a generic shape can be heated and molded onto the inner surface of the rigid prosthetic socket 150 to form the formed flexible inner socket 100. The heating temperature can be in a range from about 250° F. to about 350° F. The flexible inner socket 100 is formed on the inner surface of the prosthetic socket 150. The flexible inner socket 100, after heated and formed, therefore can have a shape matching the contour of the inner surface of the rigid prosthetic socket 150 and the anatomical shape of the residual limb 190.

In one embodiment, the flexible inner socket 100 can be attached or releasably configured on an inner surface of the prosthetic socket 150 to provide an overall inner circumference reduction of the prosthetic socket 150 based on the thickness 130 of the flexible inner socket 100. With the inner circumference reduction, the tissue of the residual limb 190 is compressed generally across the surface of the limb, and thereby bears the weight of the user. As the tissue has a larger surface area than the bone end and prominences of the residual limb 190 that might otherwise bear the weight against the hard surface of a rigid prosthetic socket, the inner circumference reduction via the compressible material of the flexible inner socket spreads the weight and causes a more comfortable fit of the prosthetic socket 150. Moreover, as described herein, the reduction reduces the inner circumference of the prosthetic socket in a predetermined manner based on the thickness of the flexible inner pre-socket to provide a globally reduced fit of the prosthetic socket.

In one embodiment, the flexible inner socket 100 is fabricated on the inner surface the prosthetic socket 150, the inner circumference reduction can be precisely controlled. In some embodiments, the inner circumference reduction is controlled by controlling the thickness 130 of the flexible inner socket 100 (or the thickness of the flexible inner pre-socket that is used to form the flexible inner socket 100). The thickness 130 of the flexible inner socket 100 can range from about 0.5 millimeters (mm) to about 8 mm or greater, or any sub-range within. The thickness 130 can be determined to provide an inner circumference reduction for tightening the fit on the residual limb ranging from about 3% to about 8% or sub-ranges within.

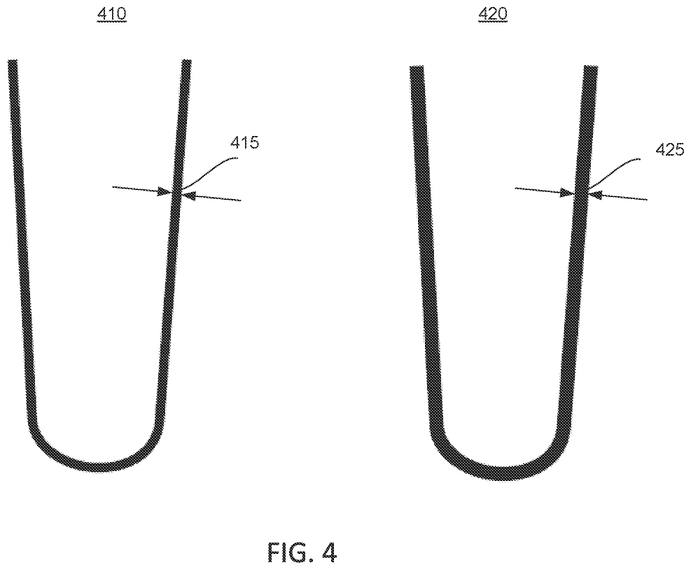

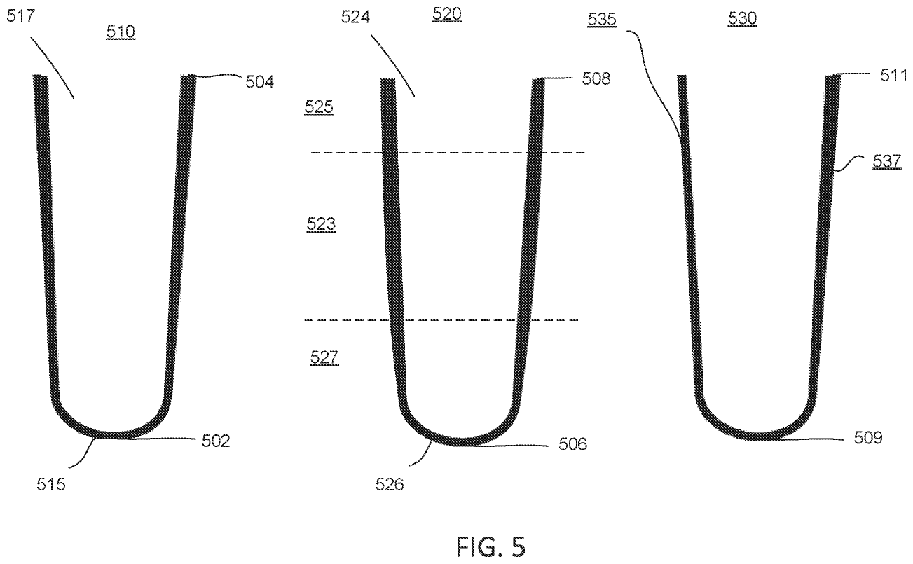

In some embodiments, the thickness 130 is the same for different portions of the flexible inner socket 100 so that the flexible inner socket 100 can provide a uniform inner circumference reduction. In other embodiments, the thickness 130 is different at different locations of the flexible inner socket 100, and the inner circumference reduction for different portions of the rigid prosthetic socket is thereby different, e.g., the thickness can be a variable thickness or any combination of thickness. The thickness 130 can be determined based on characteristics of the prosthetic socket 150 (e.g., dimensions, shapes, etc.), characteristics of the residual limb 190, characteristics of the user (e.g., weight, HMI, shape and/or dimensions of the residual limb of the patient, water retention, medical conditions, etc.), combinations of any of the same and the like. Additional features and embodiments surrounding the thickness 130 of the flexible inner socket 100 is described herein and with reference to FIGS. 4 and 5.

In one embodiment, after the flexible inner socket 100 is formed, the opening 110 of the flexible inner socket 100 is trimmed to match the contour of the opening 180 or substantially match the contour of the opening 180 of the prosthetic socket 150, The opening 110 and the material of the flexible inner socket 100 can also be configured to extend above the opening 180 in a similar contour of the opening 180 of the prosthetic socket 150 as shown in FIG. 1B. Optionally and/or alternatively, the material of the flexible inner socket 100 extending above the prosthetic socket 150 can be folded over the opening 180 of the rigid prosthetic socket 150.

In one embodiment, the flexible inner socket 100 is less rigid than the prosthetic socket 150 so that it can provide a softer edge to the prosthetic socket 150 for absorption of one or more of impact, force, stress and to provide comfort to the user. For example, the flexible inner socket 100 can have a rigidity expressed with a Shore A-type durometer value in a range from about 50 to about 90.

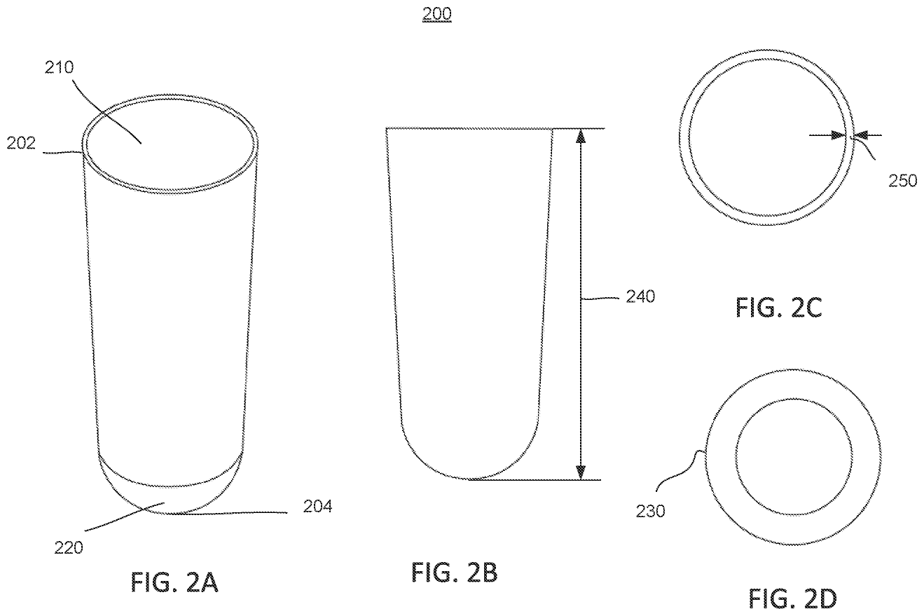

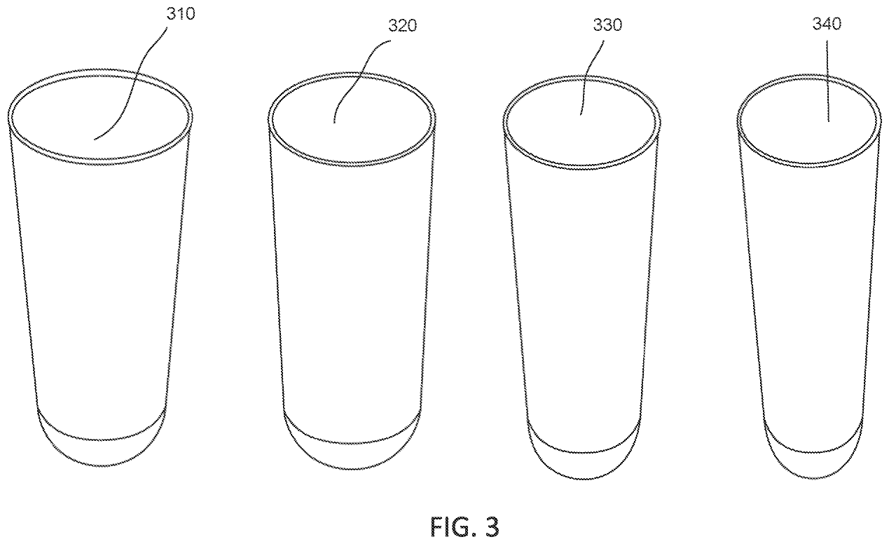

FIG. 2A is a perspective view of a flexible inner pre-socket, in accordance with another embodiment. FIG. 2B is a cross-sectional view of the flexible inner pre-socket, in accordance with another embodiment. FIG. 2C is a top view of the flexible inner pre-socket, in accordance with another embodiment. FIG. 2D is a bottom view of the flexible inner pre-socket, in accordance with another embodiment.

Referring to FIGS. 2A-2D, a perspective view of a flexible inner pre-socket 200, which can be used to form a flexible inner socket, such as the flexible inner socket 100, by being molded onto the inner surface of a prosthetic socket 150, being molded onto a residual limb of a patient, being molded on a model of a residual patient, combinations of the same or the like. The flexible inner pre-socket 200 can be formed by using a thermoformable material described herein. The thermoformable material can have one or more of the following physical properties: a Shore A-type durometer in a range from about 50 to about 90 or Shore D-type durometer in a range from about 0 to 50, a density in a range from about 0.8 to about 1.2 g/cm³, flexural Modulus in a range of about 20 to about 35 MPa, a Tensile Modulus in a range of about 3 to about 10 MPa, a tensile Strength in a range of about 4 to about 10 MPA, a tear strength of about 30 to about 50 kN/m, an elongation at break in a range from about 300% to about 800%, and a forming point temperature in a range from about of about 170° F. to about 350° F. Some suitable examples of a thermoformable material include one or more of a thermoplastic elastomer material, a thermoplastic polyurethane (TPU) material, a thermoplastic polyurethane foam material, a thermoplastic vulcanizate (TPV) material, a rubber material, an ultra-low density polyethylene (ULDPE) material, an ethylene vinyl acetate (EVA) material, a styrene material and blends of the same.

In one embodiment, the flexible inner pre-socket 200 can be formed by using various techniques, such as injection molding, blow molding, rotational molding, 3D printing techniques (e.g., fused deposition modeling, selective laser sintering, and stereolithography), combinations of the same and the like. In some embodiments, the flexible inner pre-socket 200 can include one or more layers, and each layer can be made from the same or different thermoformable material or thermoformable materials or entirely different materials, e.g., other types of thermoplastics.

The flexible inner pre-socket 200 has a first end 202, a second end 204, and the first end 202 has an opening 210 configured and dimensioned to receive at least a portion of a residual limb 190 of a user. The second end 204 is closed end 220 in a rounded type orientation. Optionally and/or alternatively, the second end has a hole or channel that extends through the thickness of the preformed socket 200. A cavity or partial channel is formed extending from the open first end 202 to the closed second end 204. The enclosed end 220 is opposite to the opening 210. The enclosed end 220 can have any type geometry, e.g., square, rectangle, cone, etc. The flexible inner pre-socket has an outer circumference 230, which is smaller than the inner circumference of the rigid prosthetic socket 150. A difference between the outer circumference 230 of the flexible inner pre-socket 200 and the inner circumference (not shown) of the rigid prosthetic socket 150 (not shown) may be no more than 15%. In one embodiment, the circumference 230 of the preformed socket 200 can be determined based on the inner circumference of the rigid prosthetic socket 150 based on formula (1) herein or other techniques herein.

The flexible inner pre-socket 200 has a length 240 that is equal to or larger than the corresponding length of the prosthetic socket 150, e.g., having a longer length than the prosthetic socket, thereby allowing the flexible inner socket 100 to be trimmed to a predetermined shape and geometry. In this embodiment, the flexible inner pre-socket 200 has a thickness 250 in a range from about 2 mm to about 8 mm. The thickness 250 of the preformed socket 200 can be determined based on the desired inner circumference reduction of the prosthetic socket 150.

Optionally and/or alternatively, a kit including multiple flexible inner pre-sockets 200 having different dimensions and shapes can provided to an end user to allow the end user to form multiple flexible inner sockets 100 that can provide different inner circumference reductions to the rigid prosthetic socket 100. The kit further can include instructions for use, e.g., selection and application of the different flexible inner sockets.

FIG. 3 illustrates a group of flexible inner pre-socket having different shapes and dimensions, in accordance with another embodiment.

Referring to FIG. 3, a group of flexible inner pre-sockets is generally depicted with reference to flexible inner pre-socket 310, flexible inner pre-socket 320, flexible inner pre-socket 330, and flexible inner pre-socket 340. Each of these flexible inner pre-sockets 310, 320, 330 and 340 have different shapes and dimensions.

Each of the flexible inner pre-sockets 310, 320, 330, and 340 are different embodiments of the flexible inner pre-socket 200 described with reference to FIGS. 2A-2D. Each of the flexible inner pre-sockets 310, 320, 330, and 340 can be used to form a flexible inner socket (e.g., the flexible inner socket 100) that can be attached to the inner surface of a rigid prosthetic socket (e.g., the rigid prosthetic socket 150) or arranged in a rigid prosthetic socket to provide an inner circumference reduction to the prosthetic socket. Optionally and/or alternatively, the group of flexible inner pre-sockets can include any number of flexible inner pre-sockets.

The group of four flexible inner pre-sockets 310, 320, 330, and 340 can include a different number of flexible inner pre-sockets with different dimensions, thickness, materials, lengths, and other attributes described herein. In this embodiment, flexible inner pre-sockets 310, 320, 330, and 340 have decreasing circumferences. Flexible inner pre-socket 310 has the largest circumference and flexible inner pre-socket 340 has the smallest circumference. Other dimensions, such as length, of the flexible inner pre-sockets 310, 320, 330, and 340 can also be different. The flexible inner pre-sockets 310, 320, 330, and 340 can also have different shapes, such as more or less conical shapes. The flexible inner pre-sockets 310, 320, 330, and 340 can have ends that are rounded or oblong. They can also have uniform thickness or have different thicknesses at different portions. Optionally and/or alternatively, the second end has a hole or channel that extends through the thickness of the flexible inner pre-socket.

The flexible inner pre-sockets 310, 320, 330, and 340 are used for forming flexible inner sockets of different sizes. These flexible inner sockets can provide different inner circumference reductions to the same rigid prosthetic socket or multiple rigid prosthetic sockets of different sizes. In some embodiments, a user may need a flexible inner socket of a different size as conditions of the user's residual limb change. For instance, the user's residual limb can undergo substantial changes in shape and volume during the postoperative recovery period, during the day or other times. As the shape or volume of the user's residual limb changes, the user needs a different amount of inner circumference reduction for comfortable fit of the prosthetic socket. The circumference of each of the flexible inner pre-sockets 310, 320, 330, and 340 is smaller than the inner circumference of the rigid prosthetic socket so that it can be placed into the prosthetics socket for being molding onto the inner surface of the prosthetic socket, being molded onto a residual limb of a patient, being molded on a model of a residual limb of a patient, combinations of the same or the like. In some embodiments, the difference between the circumference of each of the flexible inner pre-sockets 310, 320, 330, and 340 and the inner circumference of the rigid prosthetic socket is no more than 15% so that the flexible inner pre-sockets 310, 320, 330, and 340, after heated and stretched, can match the inner surface of the rigid prosthetic socket.

FIG. 4 illustrates a cross-sectional view of two flexible inner pre-sockets, each of which has a uniform thickness, in accordance with another embodiment.

Referring to FIG. 4, two flexible inner pre-sockets are generally depicted as 410 and 420. Each of the flexible inner pre-sockets 410 and 420 have a uniform thickness. Flexible inner pre-socket 410 has a thickness 415 and flexible inner pre-socket 420 has a thickness 420. Thickness 425 is larger than thickness 415. The thicknesses 415 and 425 can be in a range from about 2 mm to about 8 mm.

In one embodiment, two or more flexible inner pre-sockets, e.g., 410 and 420, can be used to form two flexible inner sockets of different thicknesses by molding the flexible inner pre-sockets 410 and 420 onto the inner surface of a same prosthetic socket, being molded onto a residual limb of a patient, being molded on a model of a residual limb of a patient combinations of the same or the like. The thickness of the flexible inner sockets can be the same or similar to the thicknesses 415 and 425 and the preformed sockets 410 and 420. The material of socket 410 and 420 can be the same or different.

In one embodiment, the two flexible inner pre-sockets 410 and 420 can be interchangeable and removable by a user from a prosthetic. As the flexible inner sockets have different thicknesses 415 and 425, they can provide different amounts of inner circumference reduction to the same rigid prosthetic socket for a user. For example, a user can wear the flexible inner socket formed from the flexible inner pre-socket 410 when the user's residual limb is engorged and larger (e.g., typically in the morning a residual limb can have a larger outer circumference), and use the flexible inner socket formed from the flexible inner pre-socket 420 when the user's residual limb is shrunk and fluid has been pressed out from the residual limb (e.g., typically in the afternoon a residual limb of user can have a smaller circumference as compared to the morning).

As the flexible inner pre-sockets 410 and 420 have uniform thicknesses 415 and 425, the flexible inner sockets provide uniform inner circumference reductions. The thickness of a flexible inner pre-sockets can have a non-uniform distribution to form a flexible inner socket providing a non-uniform inner circumference reduction. Optionally and/or alternatively, the second end has a hole or channel that extends through the thickness of the preformed socket.

FIG. 5 illustrates flexible inner pre-sockets having non-uniform thicknesses, in accordance with another embodiment.

Referring to FIG. 5, flexible inner pre-sockets 510, 520, and 530 are generally depicted each having non-uniform thicknesses as depicted in each flexible inner pre-socket. Each of the flexible inner pre-sockets 510, 520, and 530 can be an embodiment of the flexible inner pre-socket 200 herein or any embodiment herein. Each of the flexible inner pre-socket 510, 520, and 530 has a different thickness at different portions of the flexible inner pre-socket.

Flexible inner pre-socket 510 has a first end 502 and second end 504. The first end 502 is enclosed 515 and the second end 504 is open 517. The thickness of the flexible inner pre-socket 510 gradually increases from the first end 502 to the second end 504.

Flexible inner pre-socket 520 has a first end 506 and second end 508. The first end 506 is enclosed 526 and the second end 508 is open 524, Flexible inner pre-socket 520 has a first end region 527, a middle region 523 and a second end region 525, The first end region 527 has thickness less than the thickness in middle region 523 and the middle region 523 less than the thickness in the middle region 523 and first end region 527, The thickness in first end region 527, the middle region 523 and the second end region 525 can be in a range from about 1 mm to about 8 mm or greater.

Flexible inner pre-socket 530 has a first end 509, a second end 537, a left side portion 535 and right side portion 537. The left portion 535 of the preformed socket 530 has a smaller thickness than the right portion 537 of the preformed socket 530. The left side portion 535 can have a thickness in a range from about 2 mm to about 8 mm or greater and the right side portion 537 can have a thickness in a range from about 2 mm to about 8 mm or greater. Optionally and/or alternatively, there can be greater thickness at specified locations, such as where the bone prominences, etc. tend to contact the flexible inner socket. In addition, the thickness from a first end 509 to a second end 511 does not have to be linear in its rate of change from the first end 509 to the second end 511, e.g., the thickness can have any rate of change from a first end 509 to a second end 511, e.g., non-linear, linear or constant rate of change.

The thickness can be customized and optimized for a particular user's shape of their residual limb and features of the same, e.g., bone prominences, soft spots, infection, bruising, and the like. This customized and optimized thickness can be determined with computer scanner devices, manually with plaster molds, visually mapping, and any combination of the same or the like. This customized and optimized can also be adjusted in-situ with tools configured to remove thickness, e.g., grinder, sander and the like.

As the flexible inner pre-sockets 510, 520, and 530 have non-uniform thicknesses, they can form flexible inner sockets of non-uniform thicknesses. These flexible inner sockets, when attached to the inner surface of a rigid prosthetic socket or placed inside the rigid prosthetic socket, can provide non-uniform inner circumference reductions and cause non-uniform compression of the residual limb of a user wearing the flexible inner socket and rigid prosthetic socket. With a larger thickness of a portion of the flexible inner pre-socket, the corresponding portion of the flexible inner socket can provide a higher inner circumference reduction and the corresponding portion of the user's residual limb can be more compressed.

In some embodiments, the non-uniform distribution of the thickness of a primary socket is determined based on the conditions of the user's residual limb, such as shape, dimension, as described herein. Accordingly, the corresponding portion of the preformed socket can be made thicker. The non-uniform distribution of the thickness of a primary socket can also be determined based on characteristics of the user, such as weight, residual limb dimension, residual limb shape, tolerances and the like.

In one embodiment, to form a flexible inner socket from a flexible inner pre-socket, the flexible inner pre-socket is heated to a temperature so that the flexible inner pre-socket becomes a formable flexible inner pre-socket. The formable flexible inner pre-socket is placed in the prosthetic and molded onto the inner surface of the prosthetic socket such that the outer circumference of the formable flexible inner pre-socket substantially follows the contours of the inner surface of the prosthetic socket, thereby reducing the inner circumference of the prosthetic socket in a predetermined manner based on the thickness of the flexible inner pre-socket to provide a globally reduced fit of the prosthetic socket. As described herein, the molding of the preformed socket can be done through inserting pressure, force, e.g., air, vacuum pressure or other external pressure can be used to apply the pressure. Optionally and/or alternatively, the heated flexible inner pre-socket can be molded onto a residual limb of a patient, molded onto a model of a residual limb of a patient as described herein.

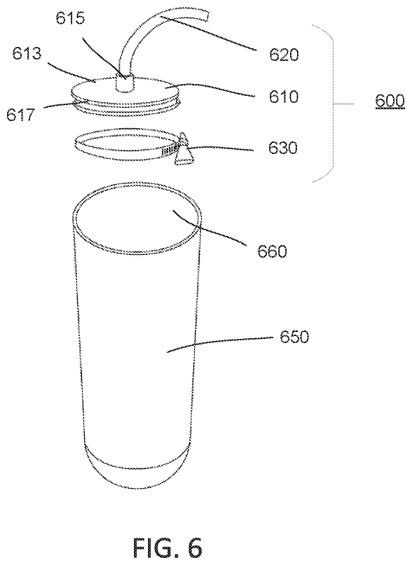

FIG. 6 illustrates an air inlet assembly used for molding a flexible inner pre-socket, in accordance with another embodiment.

Referring to FIG. 6, an air inlet assembly 600 is used for molding a flexible inner pre-socket 650. The flexible inner pre-socket 650 is the flexible inner pre-socket as described herein, e.g., in FIGS. 2A-2D. The flexible inner pre-socket 650 has a closed first end 602 and open second end 604. The second end 604 has an opening 660. The air inlet assembly 600 can be arranged over the opening 660 and seal the second end 604.

The air inlet assembly 600 includes a lid 610, a hose 620, and a clamp 630. In other embodiments, the air inlet assembly 600 can include different components that have similar functions. The flexible inner pre-socket 650 can also be made with an enclosed top that has a small opening for the hose 650 to insert into. The lid 610 seals the opening 660 of the flexible inner pre-socket 650. The lid 610 includes a lid cover 613, a valve 615, and a socket connector 617. The lid cover 613 is configured to be placed on the opening 660 of the flexible inner pre-socket 650.

The lid 610 has a top portion or cover 613 formed with a hole at a central area of the lid cover 613. The hole can include a valve 615, e.g., one-way valve, for controlling air flow. The valve 615 has a shape of a hollow cylinder. The valve 615 and the hole in the lid cover 613 provide an air channel to the flexible inner pre-socket 650. The socket connector 617 is coupled to the lid cover 613 and can be inserted into the flexible inner pre-socket 650 to make the flexible inner pre-socket 650 airtight. The socket connector 617 can be in a shape of a ring and can be made of rubber or other flexible materials. The socket connector 617 can have a top portion that is coupled to the lid cover 613 and a bottom portion that has a larger diameter than the top portion.

In one embodiment, the hose 620 is coupled to the valve 615 of the lid 610. One end of the hose 620 is inserted into the valve 615. Another end of the hose 620 can be connected to a pump that pumps air into the preformed socket 650 through the hose 620.

The clamp 630 clamps the portion of the flexible inner pre-socket 650 that encloses the socket connector 617 of the lid 610 to hold the lid 610 to the flexible inner pre-socket 650, particularly the socket connector 617 of the lid 610, with the flexible inner pre-socket 650.

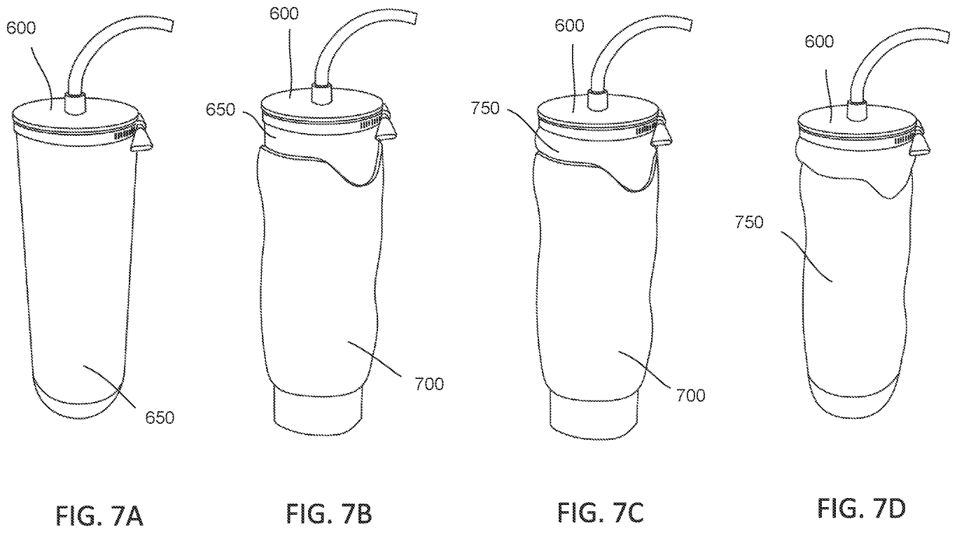

FIGS. 7A-7D illustrate a process of molding a flexible inner pre-socket onto an inner surface of a rigid prosthetic socket by using the air inlet assembly, in accordance with another embodiment.

Referring to FIGS. 7A-7D, a flexible inner pre-socket 650 is molded or arranged onto an inner surface of a prosthetic socket 700 by using the air inlet assembly 600 of FIG. 6. The air inlet assembly 600 is installed on the flexible inner pre-socket 650 and seals the opening 660 of the flexible inner pre-socket 650.

Referring to FIG. 7B, in this step, the flexible inner pre-socket 650 is heated to a temperature in a range from about 200° F. to about 280° F. The flexible inner pre-socket 650 becomes pliable due to the heating. The heated preformed socket 650 is placed into the rigid prosthetic socket 700.

Referring to FIG. 7C, in this step, air is inserted into the flexible inner pre-socket 650 by using a pump, e.g., a hand ball pump, electric pump or the like to a molding pressure. The molding pressure may be any pressure from about 1 psi to about 8 psi or greater. The pressure inflates the preformed socket 650 and presses the flexible inner pre-socket 650 against the inner surface of the prosthetic socket 700. The air utilized may be heated air, cooled air or a combination throughout process.

The flexible inner pre-socket 650 is molded onto the inner surface of the rigid, prosthetic socket 700 by the internal pressure of the flexible inner pre-socket, thereby substantially conformally forming the flexible inner socket 750 to the prosthetic socket. During the molding, the flexible inner pre-socket 650 is stretched and shaped to fit the contour of the inner surface of the rigid prosthetic socket 700. The flexible inner socket 750 has a shape that matches or substantially matches the contours of the inner surface of the prosthetic socket 700. The thickness of the flexible inner pre-socket 650 may not change or minimally changes during the molding process. The flexible inner pre-socket 650 may be heated during the insertion of the air, Referring to FIG. 7D, in this step, the flexible inner socket 750 has been formed and can now be removed from the prosthetic socket 700.

In one embodiment, the flexible inner pre-socket 650 may expand out of the rigid prosthetic socket 700 during the molding process. Also, the rigid prosthetic socket 700 may have a smaller length than the flexible inner pre-socket 650, and a portion of the flexible inner pre-socket 650 is not enclosed in the rigid prosthetic socket 700 as shown in FIG. 7B. This portion of the flexible inner pre-socket 650 is not constrained by the rigid prosthetic socket 700 and may expand in an undesired manner during the inflation, which can result in the flexible inner socket 750 having an undesired shape. Wraps can be used during the molding process to prevent undesired expansion of the flexible inner pre-socket 650.

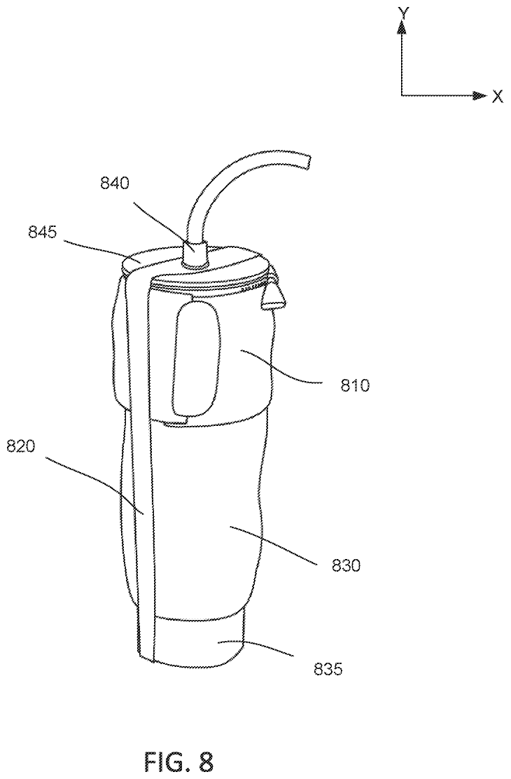

FIG. 8 illustrates wraps used for constraining a flexible inner pre-socket during molding of the flexible inner pre-socket, in accordance with another embodiment.

Referring to FIG. 8, illustrate wraps 810 and 820 used for constraining a preformed socket during molding of the flexible inner pre-socket. The flexible inner pre-socket 830 can be molded onto the inner surface of a rigid prosthetic socket 830 using the process described in conjunction with FIGS. 7A-7D.

In this embodiment, a flexible inner pre-socket is placed in the prosthetic socket 830. The wraps 810 and 820 constrain the preformed socket during its inflation by the inserted air. The wraps 810 and 820 can prevent the preformed socket from expanding out of the opening of the prosthetic socket 830. Optionally and/or alternatively, one of either wrap 810 and 820 may be used. The wraps 810 and 820 can be made from Velcro bands, fabric, polymer or other materials that provide the proper amount of constraint.

The wrap 810 circumferentially wraps a portion of the prosthetic socket 830 to constrain expansion of the preformed socket. The wrap 810 can also circumferentially wraps a portion of the preformed socket, such as a portion of the flexible inner pre-socket that is not enclosed in the rigid prosthetic socket 830. The wrap 810 reduces circumferential expansion of the flexible inner pre-socket.

The wrap 820 wraps the prosthetic socket 830 and is arranged over the top surface of the lid 845 of the air inlet assembly 840 the sides of the rigid prosthetic socket 830, and the bottom of the base 835 of the prosthetic socket 830. The flexible inner pre-socket is enclosed within the lid 845 of the air inlet assembly 840 and the prosthetic socket 830, so that the wrap 820 prevents the preformed socket from expanding vertically along a y-axis.

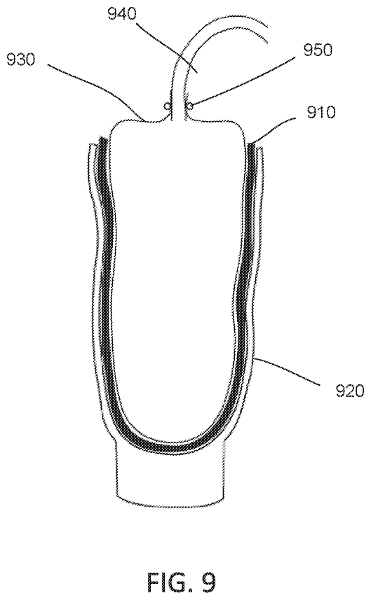

FIG. 9 illustrates a process of molding a flexible inner pre-socket onto an inner surface of a rigid prosthetic socket by using an air bag, in accordance with another embodiment.

Referring to FIG. 9, a process of molding a flexible inner pre-socket 910 onto an inner surface of a rigid prosthetic socket 920 by using an air bag 930 is generally described. The flexible inner pre-socket 910 can be the flexible inner pre-socket 200 or any flexible inner pre-socket described herein. The flexible inner pre-socket 920 can be a rigid prosthetic socket 150 or any prosthetic socket described herein.

In a first step, the flexible inner pre-socket 910 is placed in the prosthetic socket 920. Next, the airbag 930 is placed in the flexible inner pre-socket 910. The airbag 930 is coupled to a hose 940, through which air source or pump can be coupled to the airbag 930. The hose 940 is coupled to a valve 950 that controls flow of the air. For instance, air can be inserted into the air bag 930 when the valve 950 is open and is prevented from flowing into the air bag when the valve 950 is closed.

The flexible inner pre-socket 910 is heated before and/or while the air is inserted into the air bag 940 with heated air or other heat source. Due to the heating, the flexible inner pre-socket 910 is pliable and configured to move from a first orientation to a second orientation. The airbag 940 is inflated by the air and press the flexible inner pre-socket 910 against the inner surface of the rigid prosthetic socket 920 to mold the material of the flexible inner pre-socket 910 to a contour of the inner surface of the prosthetic socket 920. The molded shape is maintained when it cools down to room temperature as the material becomes less pliable and retains or substantially retains the heated memory shape. After it is cooled the pressure or air source is released.

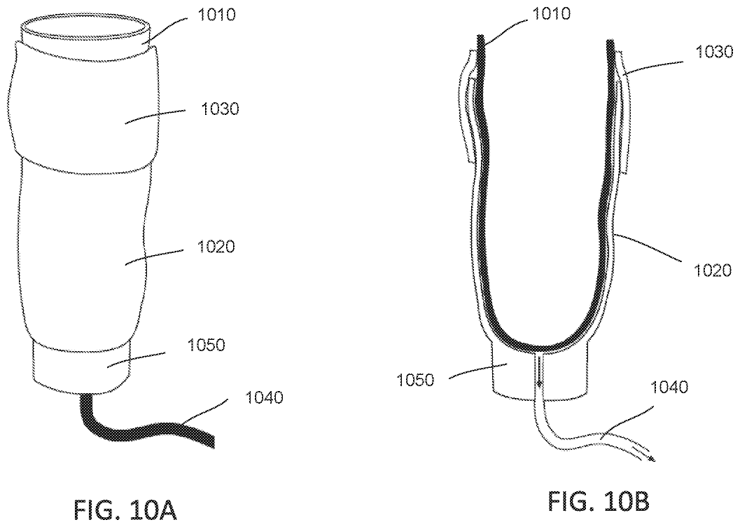

FIG. 10A is a perspective view of a system of molding a flexible inner pre-socket onto an inner surface of a rigid prosthetic socket by vacuum, in accordance with another embodiment. FIG. 10B is a cross-sectional view of the system, in accordance with another embodiment.

Referring to FIGS. 10A-110B, a flexible inner pre-socket 1010 is molded onto an inner surface of a prosthetic socket 1020 by vacuum pressure form a vacuum source with an aid of a sleeve 1030 and a vacuum hose 1040. The flexible inner pre-socket 1010 is placed into the prosthetic socket 1020. A portion of the flexible inner pre-socket 1010 is enclosed by the rigid prosthetic socket 1020 and another portion of the flexible inner pre-socket 1010 is outside the rigid prosthetic socket 1020. For the portion of the flexible inner pre-socket 1010 that is enclosed by the prosthetic socket 1020, there is a gap between the outer surface of the flexible inner pre-socket 1010 and the inner surface of the prosthetic socket 1020. The gap is sealed by, the sleeve 1030. As shown in FIG. 1013, the sleeve 1030 is applied on the rigid prosthetic socket 1020 and the portion of the flexible inner pre-socket 1010 that is outside the rigid prosthetic socket 1020.

A vacuum hose 1040 is installed on the base 1050 of the rigid prosthetic socket 1020. The vacuum hose 1040 provides a channel to the gap between the flexible inner pre-socket 1010 and the rigid prosthetic socket 1020. The vacuum hose 1040 is coupled to a vacuum source (not shown). In FIG. 10C, the air in the gap between the flexible inner pre-socket 1010 and the rigid prosthetic socket 1020 is pumped out from the gap by the vacuum pump to create a vacuum, or near vacuum, in the gap. The flexible inner pre-socket 1010 is pliable during creation of the vacuum by application of heat or previous application of heat that has been applied or is being applied to the flexible inner pre-socket 1010. Due to the vacuum pressure, the flexible inner pre-socket 1010 is pulled toward the inner surface of the rigid prosthetic socket 1020 to mold onto the inner surface of the rigid prosthetic socket 1020 and to form a flexible inner socket.

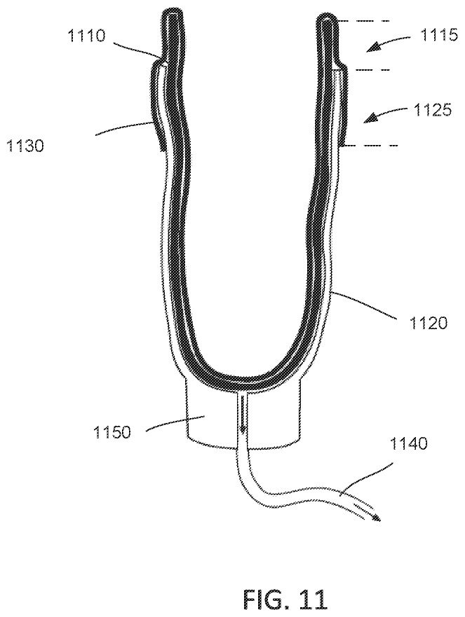

FIG. 11 illustrates another system of molding a flexible inner pre-socket onto an inner surface of a rigid prosthetic socket by vacuum, in accordance with another embodiment.

Referring to FIG. 11, a flexible inner pre-socket 1110 is molded onto an inner surface of a prosthetic socket 1120 by vacuum. The flexible inner pre-socket 1110 is placed in the prosthetic socket 1120 and a top portion 1115 of the flexible inner pre-socket 1110 extends outside the opening of the prosthetic socket 1120. A flexible bladder 1130 is arranged around the flexible inner pre-socket 1110 and extends outside the opening of the prosthetic socket 1120 and is folded around the top portion 1125 of the prosthetic socket 1120 to cover a portion of the prosthetic socket 1120.

In this embodiment, the flexible bladder 1130 wraps into the flexible inner pre-socket 1110 and over the top portion 1115 of the flexible inner pre-socket 1110 and the top portion 1125 of the rigid prosthetic socket 1120. As shown in FIG. 11, the flexible bladder 1130 covers the inner surface of the flexible inner pre-socket 1110, the outer surface of the top portion 1115 of the flexible inner pre-socket 1110, and the outer surface of the top portion 1125 of the prosthetic socket 1120. The flexible bladder 1130 provides a seal the gap between the flexible inner pre-socket 1110 and the prosthetic socket 1120. The flexible bladder is stretched over the top portion 115 and 1125.

The vacuum hose 1140 connects to a vacuum source (not shown) and is operated to create a vacuum pressure, or near vacuum pressure, in the gap. The vacuum hose 1140 can be the same as the vacuum hose 1040. Due to the vacuum pressure, the preformed socket 1110 is molded onto the inner surface of the rigid prosthetic socket 1120 and to form a flexible inner socket. Optionally and/or alternatively, the second end of the flexible inner pre-socket 1110 has a hole or channel that extends through the thickness of the flexible inner pre-socket. The hole or channel (not shown) allows the vacuum pressure to communicate directly with the preformed socket through the hole or channel. In this embodiment, the flexible inner pre-socket is heated prior to inserting into the prosthetic socket to a thermoformable temperature as described herein. The flexible inner pre-socket is allowed to cool and the vacuum source is turned off.

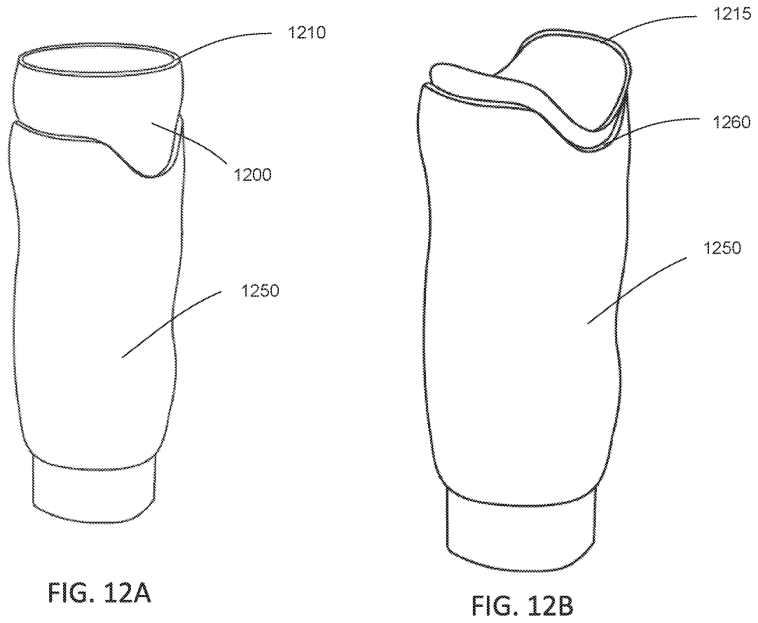

FIGS. 12A-12B illustrates a process of trimming a flexible inner socket 1200, in accordance with another embodiment.

Referring to FIG. 12A, a first flexible inner socket 1200 having a top edge 1210 untrimmed extending above a prosthetic socket 1250 is shown. In FIG. 1213, the top edge 1210 is trimmed to top edge 1215 to follow the top edge 1260 of the prosthetic socket 1250. The flexible inner socket 1200 is used to provide inner circumference reduction to a rigid prosthetic socket 1250. The flexible inner socket 1200 is formed by molding a preformed socket onto the inner surface of the rigid prosthetic socket 1250 as described in embodiments herein.

More specifically, the flexible inner socket 1200 has a top edge 1210 that forms the opening of the flexible inner socket 1200. The rigid prosthetic socket 1250 has a top edge 1260 that forms the opening of the prosthetic socket 1250. An embodiment of the flexible inner socket 1200 can be the flexible inner socket 100, and an embodiment of the rigid prosthetic socket 1250 can be the prosthetic socket 150.

Before the trimming, the flexible inner socket 1200 has a flat top edge 1210, as shown in FIG. 12A. The trimming of the flexible inner socket 1200 includes trimming the top edge 1210 to make it match the top edge 1260 of the prosthetic socket 1250. As shown in FIG. 12B, the trimmed top edge 1215 of the flexible inner socket 1200 has a contour that is the same as or similar to the contour of the top edge 1260 of the rigid prosthetic socket 1250. In some embodiments, the length of the flexible inner socket 1200 after the trimming is larger than the length of the rigid prosthetic socket 1250 so that the top edge 1215 of the flexible inner socket 1200 can be bent, e.g., while being heated by a heat gun, to cover the top edge 1260 of the rigid prosthetic socket 1250. The flexible inner socket 1200 can be softer than the rigid prosthetic socket 1250 so that it is more comfortable for the user's residual limb to contact the flexible inner socket 1200 than the prosthetic socket 1250.

Even though the flexible inner socket 1200 is placed in the prosthetic socket 1250 in FIGS. 12A-1213, the flexible inner socket 1200 can be removed from the prosthetic socket 1250 before the trimming.

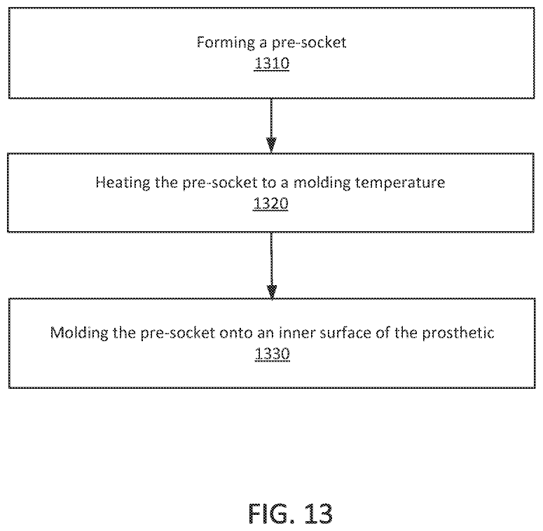

FIG. 13 illustrates a flow chart of a process for fabricating a flexible inner socket, in accordance with another embodiment.

Referring to FIG. 13, the process of fabricating a flexible inner socket is generally shown. The flexible inner socket is used for reducing an inner circumference of a prosthetic socket, e.g., rigid, socket, that has previously been formed to fit a residual limb of a user. The process may include different or additional steps than those described in conjunction with FIG. 13 in some embodiments or perform steps in different orders than the order described in conjunction with FIG. 13.

A flexible inner pre-socket is formed (step 1310) with a material. The flexible inner pre-socket is formed with an opening and an enclosed end. The enclosed end is opposite the opening. A circumference of the body is smaller than the inner circumference of the prosthetic socket. For example, the circumference of the body can be up to 15% smaller than the inner circumference of the rigid prosthetic socket.

In some embodiments, the thermoformable material of the flexible inner pre-socket is a material, e.g., polymer, having one or properties: a Shore A-type durometer in a range from about 55 to about 85, an elongation of at least 300%, and a melting point below about 140° C. The material can be a thermoplastic elastomer material, a thermoplastic polyurethane (TPU) material, a thermoplastic polyurethane foam material, a thermoplastic vulcanizate (TPV) material, a rubber material, an ultra-low density polyethylene (ULDPE) material, an ethylene vinyl acetate (EVA) material, a styrene material, and blends of the same, combination of the same or the like. The flexible inner pre-socket can be formed through one more of injection molding, rotational molding, 3D printing (e.g., fused deposition modeling, selective laser sintering, or stereolithography), blow molding, combinations of the same and the like.

In some embodiments, a thickness of the flexible inner pre-socket is predetermined, e.g., based on the dimensions of the prosthetic socket, shape and dimensions of the user's residual limb, the user's health conditions, or some combination thereof. In one embodiment, the pre-socket has a uniform thickness. In another embodiment, different portions of the flexible inner pre-socket have different thicknesses as described herein.

In step 1320 the flexible inner pre-socket is heated to a temperature between 250° F. to 350° F. The flexible inner pre-socket becomes pliable due to the heat. After the heating 1320, the flexible inner socket is formed 1330 in one of the embodiments described herein. In one embodiment, the flexible inner pre-socket is formed into a flexible inner socket by molding the flexible inner pre-socket onto the inner surface of the rigid prosthetic socket. Optionally and/or alternatively, the flexible inner pre-socket is molded onto a residual limb of a patient. In another embodiment, the flexible inner pre-socket is molded on a model of a residual limb of a patient.

In one embodiment, the rigid prosthetic socket has been formed to fit a residual limb of a user. The rigid prosthetic socket can be formed on the residual limb of the user or on a model of the residual limb so that the inner surface of the rigid prosthetic socket has a contour and dimensions matching the anatomical shape of the residual limb. No global reduction was applied to the rigid prosthetic socket during the forming of the rigid prosthetic socket.

In some embodiments, the flexible inner pre-socket is molded onto the inner surface of the rigid prosthetic socket by inserting air into the flexible inner pre-socket. The flexible inner pre-socket can be sealed with an air inlet assembly to form an enclosed space. The sealed flexible inner pre-socket is placed in the rigid prosthetic socket. Air is inserted into the flexible inner pre-socket to inflate the flexible inner pre-socket. The air can press the flexible inner pre-socket against the inner surface of the rigid prosthetic socket to form the flexible inner socket.

In yet some other embodiments, the flexible inner pre-socket is molded onto the inner surface of the prosthetic socket by creating vacuum pressure in a gap between the flexible inner pre-socket and the rigid prosthetic socket. The vacuum pressure can pull the flexible inner pre-socket toward the inner surface of the rigid prosthetic socket to form the flexible inner socket. The outer surface of the formed flexible inner socket has a contour and dimensions matching the contour and dimensions of the inner surface of the rigid prosthetic socket. The flexible inner socket can then be cooled down, e.g., to room temperature.

The flexible inner socket can be trimmed after being removed from the rigid prosthetic socket. For instance, the top edge of the flexible inner socket is trimmed to match the top edge of the rigid prosthetic socket. The flexible inner socket can be placed into the rigid prosthetic socket for use by the user.

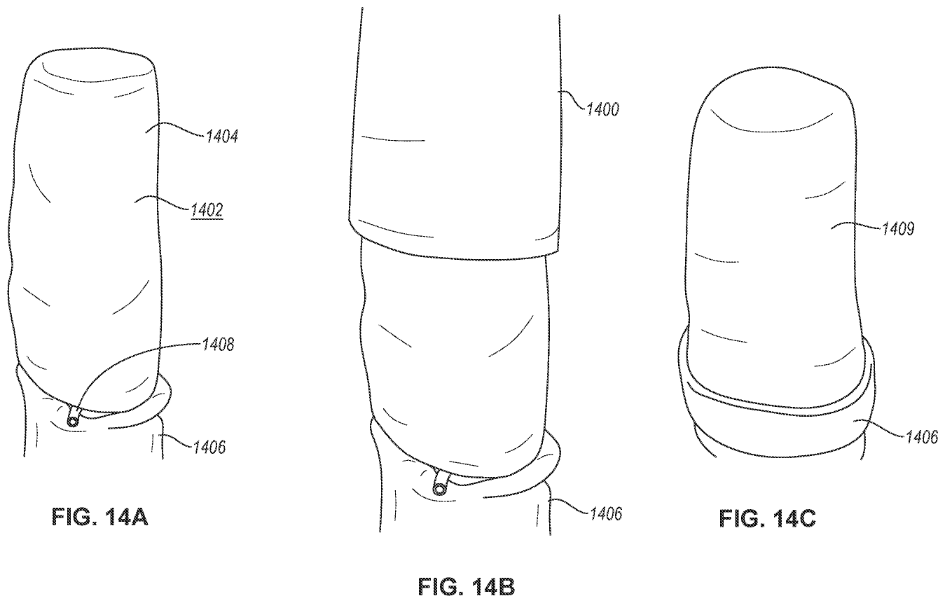

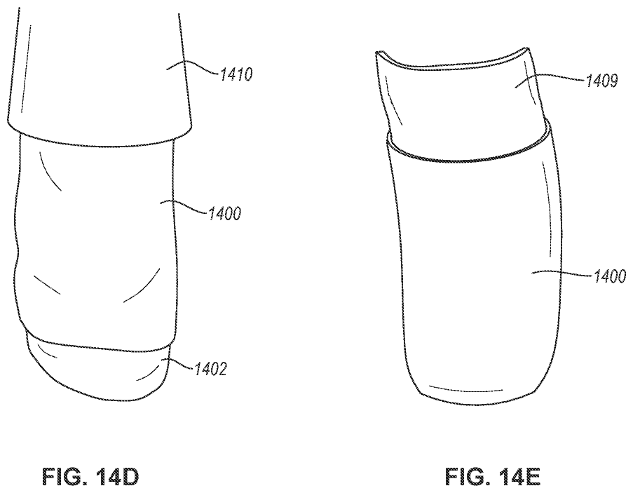

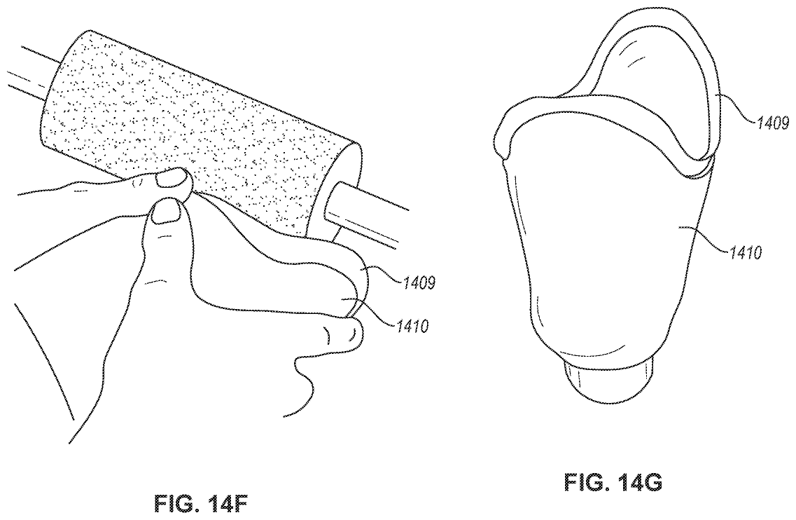

FIG. HA illustrates a mold configured to mimic a residual limb of a patient; FIG. 14B illustrates a process for molding a flexible inner pre-socket onto a mold of FIG. 14A. FIG. 14C illustrates a molded flexible inner pre-socket on a mold of FIG. 14A. FIG. 14D illustrates a process of forming a prosthetic socket onto a flexible inner socket in a prosthetic socket according to another embodiment. FIG. 14E illustrates a flexible inner socket and a prosthetic socket according to another embodiment. FIG. 14F illustrates a process of finishing the edge of a flexible inner socket in a prosthetic socket according to another embodiment. FIG. 14G illustrates a trimmed flexible inner socket arranged in a trimmed prosthetic socket according to another embodiment.

Referring to FIG. 14A-14G, a process of molding a flexible inner pre-socket 1400 onto a model or positive mold 1402 of residual limb of a user. The positive mold 1402 may be constructed as known in the art or described herein. The positive mold 1402 has an outer surface 1404 that matches or mimics the surface and contours of a residual limb of a patient as shown in FIG. 14A. In a preferred embodiment, the positive model 1402 has been globally reduced. The flexible inner pre-socket 1400 is heated to a molding temperature and molded onto the positive mold 1402 as shown in FIG. 14B. The heated flexible pre-socket is pulled down using gloved hands over the positive mold 1402. A sleeve 1406 is rolled up and vacuum is applied until the heated flexible pre-socket is cooled as shown in FIG. 14C. Optionally, a stockinette is pulled over the flexible inner pre-socket and the vacuum is applied through a vacuum hose 1408. This process takes about one minute and cooling takes a few more or a bit longer. A flexible inner socket 1409 is now formed from the flexible inner pre-socket 1400 on the positive mold 1402.

A prosthetic socket 1410 is heated to a molding temperature of and pulled over the formed flexible inner socket 1409 as shown in FIG. 14D. The prosthetic socket 1410 and heating temperatures required for forming are described with reference to U.S. Pat. Nos. 15/914.480 and 16/516,199, both of which are hereby incorporated by reference as if fully set forth herein. The prosthetic socket 1410 is now allowed to cool.

The prosthetic socket 1410 and the flexible inner socket 1409 are now removed from the positive mold 1402 as shown in FIG. 14E. The prosthetic socket 1410 and the flexible inner socket 1409 are now trimmed, grinded and/or buffed as shown in FIG. 14F to remove a predetermined amount of material and into a desired geometry. The trimmed, grinded and/or buffered prosthetic socket 1410 and flexible inner socket 1409 are shown in FIG. 14B. Optionally, the edges of either the flexible inner socket 1409 and prosthetic socket 1410 can be heated with a heat gun and flared and shaped as desired.

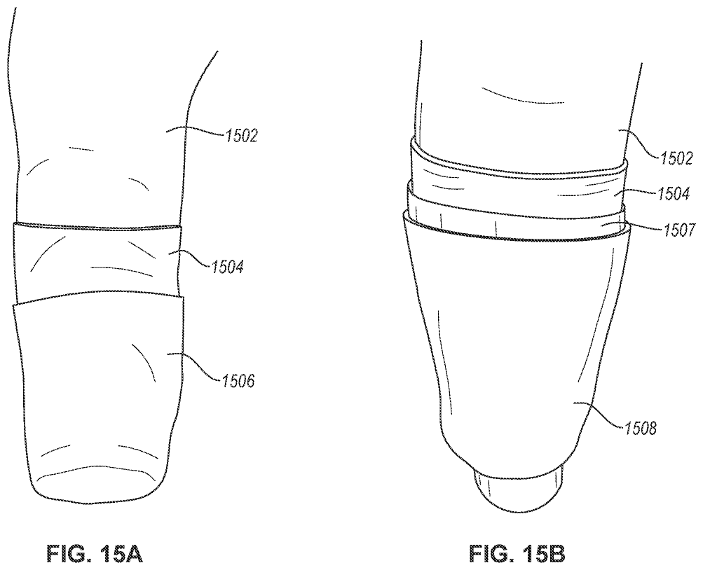

FIG. 15A illustrates a process of molding a flexible inner pre-socket on a residual limb of a patient according to another embodiment. FIG. 15B illustrates a process of molding a prosthetic socket on a flexible inner socket on a residual limb of a patient according to another embodiment.

Referring to FIGS. 15A-15B, a process of molding a flexible inner pre-socket 1506 onto a residual limb of a user is now described. The residual limb 1502 includes a protective thermal barrier configured to protect a patient from a heated flexible inner pre-socket and/or a heated prosthetic socket, e.g., a sock. The flexible inner pre-socket 1560 is heated to a molding temperature and molded onto the residual limb as shown in FIG. 15A. The heated flexible pre-socket 1506 is pulled onto the residual limb 1502 using gloved hands. A flexible inner socket 1507 is now formed from the flexible inner pre-socket 1506 directly onto a residual limb 1502 of a patient.

A prosthetic socket 1508 is heated to a molding temperature of and pulled over the formed flexible inner socket 1507 as shown in FIG. 15B. The prosthetic socket 1508 and heating temperatures required for forming are described with reference to U.S. Pat. Nos. 15/914,480 and 16/516,199, both of which are hereby incorporated by reference as if fully set forth herein. The prosthetic socket 1508 is now allowed to cool.

The prosthetic socket 1508 and the flexible inner socket 1507 are now removed from the residual limb and trimmed, grinded and/or buffed as described herein. Optionally, the edges of either the flexible inner socket 1507 and prosthetic socket 1508 can be heated with a heat gun and flared as desired.

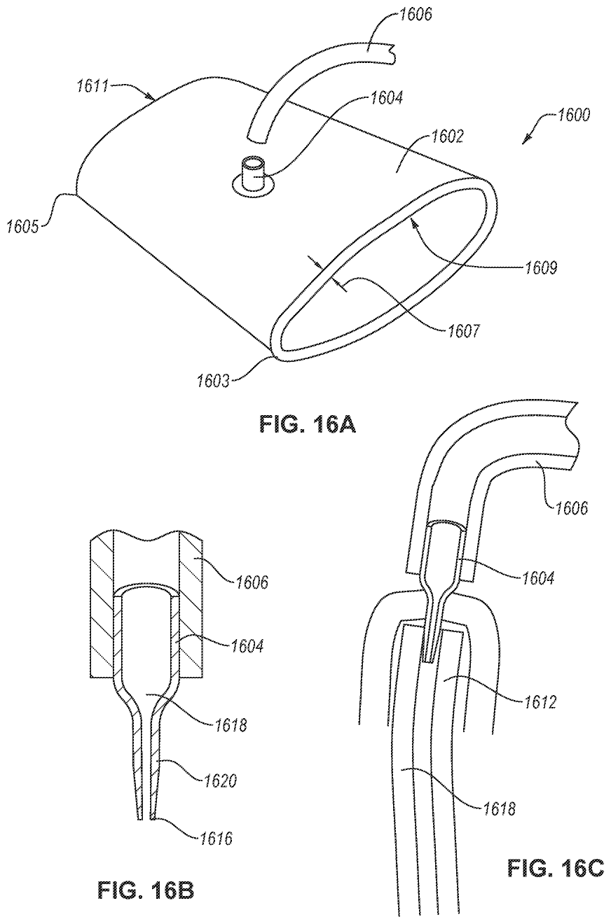

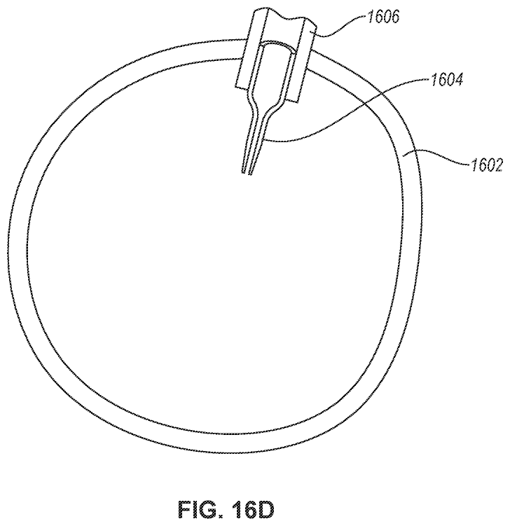

FIG. 16A illustrates an assembly used for molding a flexible inner pre-socket, in accordance with another embodiment. FIG. 16B illustrates a partial cross-sectional view of a portion of FIG. 16A. FIG. 16C illustrates a partial cross-sectional view of a portion of FIG. 16A on a portion of the flexible inner pre-socket. FIG. 16D illustrates a partial cross-sectional view of a portion of FIG. 16A on a portion of the flexible inner pre-socket.

Referring to FIGS. 16A-16D, a molding apparatus is generally depicted with reference to number 1600. The molding apparatus 1600 includes an impermeable elastic sleeve 1602 having a first end 1603, a second end 1605, a thickness 1607 and first end opening 1609 that extends from the first end 1603 through the second end 1605 to an open second end 1611, The elastic sleeve 1602 can include one of the following materials: A silicone material, an elastomeric gel, and elastomeric gel sleeve with fabric covering such as used by amputees to hold and seal a prosthetic socket onto a limb, rubber, latex or other impermeable stretchy material. The apparatus 1600 also includes a port 1604 extending through a thickness of the sleeve 1602. The port 1604 includes a first end 1614, a second end 1616, and channel or lumen 1618 extending from the first end 1614 to the second end 1616. The first end 1614 has a wider diameter than the second end 1620. The second end portion 1620 has a taper portion going from a wider diameter to narrower diameter. The first end 1614 is configured to receive a hose 1606. The hose 1606 is configured to be connected to a vacuum source (not shown).

Referring to FIG. 16C, in operation, a flexible inner pre-socket 1618 is arranged into a rigid prosthetic socket 1612 after being heated to a formable temperature in an range from about 275° F. to about 325° F. The sleeve 1602 is arranged over an end portion of the prosthetic socket, so the end portion 1620 of the port 1604 is arranged between an outer wall of flexible inner pre-socket 1618 and the inner wall of the prosthetic socket 1612 and a vacuum source operated in range from about 15-25 InHg of vacuum. The vacuum source creates a negative pressure the pulls the heated stretchable flexible inner pre-socket 1618 onto the inner wall of the prosthetic socket 1612, thereby molding it onto the inner wall. The molded shape is maintained when it cools down to room temperature as the material becomes less pliable and retains or substantially retains the heated memory shaped.

### EXAMPLES

Without intending to limit the scope of the invention, the following examples illustrate how various embodiments of the invention may be made and/or used.

### Example 1

This example illustrates the manufacture of flexible inner socket according to an aspect of the invention. A flexible inner pre-socket was made with a thermoformable polyurethane material GLS Versaflex CE 3115 that was a Shore A-type durometer of about 65 A. It was injection molded on a 500 ton injection press. It was conical in shape and measured 30 cm tall, 11.5 cm diameter at the open proximal end, 94 cm in diameter at the substantially closed distal end, and was 2.5 mm thick uniformly from the proximal end to the distal end.

A rigid heat formable injection molded prosthetic socket was provided for a below-the-knee amputee as described with reference to U.S. Pat. Nos. 15/914,480 and 16/516,199, both of which are hereby incorporated by reference as if fully set forth herein. The rigid prosthetic socket was formed by heating it to 250° F. and forming it directly to the users residual limb, that had a gel/fabric liner applied, without the typical global reduction being applied. It included first end, a second end, an inner circumference. The first end including opening and the second end was substantially closed. The forming result was that the inner socket surface had contours that substantially mimic or matched contours of a residual limb of user. The rigid prosthetic socket was trimmed to size and the edges were buffed and rounded. The flexible inner pre-socket was slightly smaller externally than the rigid prosthetic socket was internally so it could be fit inside easily. The rigid prosthetic socket had a vacuum tube attached to a small opening in the distal end that was connected to an electric vacuum pump.

The flexible inner pre-socket was heated with a Nutrichef infrared circulating oven model no. PKRT97 to 250° F. for 10 minutes. It was suspended on an armature to hold it shape and became pliable yet not sticky or difficult to handle with gloves. The heated flexible inner pre-socket was inserted into the rigid prosthetic socket which was room temperature. A flexible closed end bladder was quickly dropped inside the flexible inner pre-socket and the open end was wrapped over the top and fit tightly to the outside of the rigid prosthetic socket forming a seal. The vacuum pump was immediately turned on and the resulting vacuum immediately pulled the heated pliable pre-socket to the interior shape of the rigid prosthetic socket in every detail. The flexible inner pre-socket was allowed to cool while maintaining this shape under vacuum.

A marker was used to make a trim line that extended from 1 cm to 2 cm above the edge of the rigid prosthetic socket. The formed flexible inner socket was removed and cut with shears to shape. The edge was ground with a buffing tool to be smooth and rounded. A small hole was made in the distal end for the purpose of using a vacuum suspension retention system to hold the socket to the limb. It was inserted back inside the rigid prosthetic socket and referred to as a flexible inner socket. A heat gun was used to heat the edges of it to flare them and accommodate the needs of the user.

The resulting product was a flexible inner socket globally reduced the inner circumference of the rigid prosthetic socket by about 4% in a relatively precise manner. Metal fittings, a pylon, and prosthetic foot were attached. The user donned their gel/fabric inner and stepped into the device. It is critical that the global reduction compresses the flesh and muscle in a conical fashion thereby relieving pressure to the bone end and bearing the users weight evenly on the limb. The user walked on the prosthetic for several minutes and proclaimed that it was very comfortable and supported the weight away from the bone end adequately. A proper global reduction had been achieved.

After some more use, the user had some painful pressure on the distal anterior end as can often happen with use. The area was marked and the prosthetic was removed. The prosthetic socket, being heat formable, had the area heated with a heat gun and the flexible inner socket was removed and also heated in the same area to about 225° F. It was quickly reassembled and the user stepped in. The pressure point was relieved as the limb pressed the heat softened layers out. The flexible inner socket and prosthetic socket were cooled, and the result was a very comfortable good fitting prosthetic socket that the user was able to wear successfully. Both the flexible inner socket and the prosthetic socket are readily adjustable as described herein.

The language used in the specification has been principally selected for readability and instructional purposes, and it may not have been selected to delineate or circumscribe the inventive subject matter. It is therefore intended that the scope of the disclosure be limited not by this detailed description, but rather by any claims that issue on an application based hereon. Accordingly, the disclosure of the embodiments is intended to be illustrative, but not limiting, of the scope of the disclosure, which is set forth in the following claims.

1. A flexible inner pre-socket, comprising:
an injection molded premade thermoplastic elastomer pre-socket comprising a thermoformable material, a first end, a second end, a thickness, an inner circumference, and an outer circumference, the first end having an opening, and the second end being substantially closed,
wherein the injection molded premade thermoplastic elastomer pre-socket is configured to be:
i) heated to a formable temperature so that the injection molded premade thermoplastic elastomer pre-socket becomes formable and stretchable so as to be arranged over at least a portion of a positive model of a residual leg limb to follow one or more contours of an outer surface of the positive model of the residual leg limb, or
ii) heated to a formable temperature so that the injection molded premade thermoplastic elastomer pre-socket becomes formable and stretchable so as to be arranged inside of a rigid prosthetic socket and follow contours of an inner surface of the rigid prosthetic socket, so as to globally reduce an interior volume of the rigid prosthetic socket,
wherein the injection molded premade thermoplastic elastomer pre-socket is configured to be cooled to an ambient temperature after being heated to the formable temperature such that the injection molded premade thermoplastic elastomer pre-socket becomes non-formable at the ambient temperature, and
wherein the injection molded premade thermoplastic elastomer pre-socket is injection molded in a size to stretched onto a specific positive model having a predetermined distal circumference.

2. The flexible inner pre-socket of claim 1, wherein the predetermined distal circumference is in a range from about 24 cm to 27 cm.

3. The flexible inner pre-socket of claim 1, wherein the predetermined distal circumference is in a range from about 27 cm to 30 cm.

4. The flexible inner pre-socket of claim 1, wherein the predetermined distal circumference is in a range from about 30 cm to 34 cm.

5. The flexible inner pre-socket of claim 1, wherein the predetermined distal circumference is in a range from about 34 cm to 38 cm.

6. The flexible inner pre-socket of claim 1, wherein the predetermined distal circumference is in a range from about 38 cm to 40 cm.

7. The flexible inner pre-socket of claim 1, wherein the thickness is in a range from about 2 mm to about 4 mm.

8. The flexible inner pre-socket of claim 1, wherein the thickness is not a constant thickness.

9. The flexible inner pre-socket of claim 1, wherein the thickness at a proximal end portion of the flexible inner pre-socket is thicker than the thickness at a distal end portion of the flexible inner pre-socket.

10. The flexible inner pre-socket of claim 11, wherein the injection molded premade thermoplastic elastomer pre-socket is a polymer that contains at least a portion of a slip agent comprising one or more of a silicone material and a polytetrafluoroethylene (PTFE) material or other agent that reduces a surface coefficient of friction.

11. The flexible inner pre-socket of claim 1, wherein the formable temperature is an range from about 275° F. to about 325° F.

12. The flexible inner pre-socket of claim 1, wherein the formable temperature is a temperature in an range from about 313° F. to about 317° F.

13. The flexible inner pre-socket of claim 1, wherein the injection molded premade thermoplastic elastomer socket comprises two different layers of materials.

14. The flexible inner pre-socket of claim 1, wherein the inner circumference at the first end having an opening is larger than the inner circumference at the second end.

15. The flexible inner pre-socket of claim 1, wherein the injection molded premade thermoplastic elastomer socket comprises an outer surface and an inner surface, and the outer surface comprises one or more of a textured surface, a non-polished surface, a polished surface, a patterned surface, and a corrugated surface and the inner surface comprises one or more of one or more of a textured surface, a polished surface, a patterned surface, a non-polished surface, and a corrugated surface that is imparted from a surface of an injection molding tooling.

16. The flexible inner pre-socket of claim 1, wherein the thermoformable material includes one or more of a thermoplastic elastomer (TPE) material, a thermoplastic polyurethane (TPU) material, a thermoplastic polyurethane foam material, a thermoplastic vulcanizate (TPV) material, a rubber material, an ultra-low density polyethylene (ULDPE) material, an ethylene vinyl acetate (EVA) material, a styrene material and blends of the same.

17. A flexible inner pre-socket, comprising:
an injection molded premade thermoplastic elastomer pre-socket comprising a thermoformable material, a first end, a second end, a thickness, an inner circumference at a first end portion that is larger than an inner circumference at a second end portion, and an outer circumference, the first end having an opening, and the second end being substantially closed,
wherein the injection molded premade thermoplastic elastomer pre-socket is configured to be:
i) heated to a formable temperature in a range from about 311° F. to about 320° F. so that the injection molded premade thermoplastic elastomer pre-socket becomes formable and stretchable so as to be arranged over at least a portion of a positive model of a residual leg limb and follow contours of an outer surface of the positive model of the residual leg limb, or
ii) heated to a formable temperature so that the injection molded premade thermoplastic elastomer pre-socket becomes formable and stretchable so as to be arranged inside of a rigid prosthetic socket and follow contours of an inner surface of the rigid prosthetic socket, so as to globally reduce an interior volume of the rigid prosthetic socket,
wherein injection molded premade thermoplastic elastomer pre-socket is configured to be cooled to an ambient temperature after being heated to the formable temperature such that injection molded premade thermoplastic elastomer pre-socket becomes non-formable at the ambient temperature, and
wherein the injection molded premade thermoplastic elastomer pre-socket is injected molded in a size to accommodate a specific positive model having a predetermined distal circumference in a range from about 24 cm to about 40 cm.

18. The flexible inner pre-socket of claim 17, wherein the thermoformable material includes one or more of a thermoplastic elastomer (TPE) material, a thermoplastic polyurethane (TPU) material, a thermoplastic polyurethane foam material, a thermoplastic vulcanizate (TPV) material, a rubber material, an ultra-low density polyethylene (ULDPE) material, an ethylene vinyl acetate (EVA) material, a styrene material, and blends of the same.

19. The flexible inner pre-socket of claim 18, wherein the thermoformable materials further comprises a slip agent additive.

20. A flexible inner pre-socket, comprising:
an injection molded premade thermoplastic elastomer pre-socket comprising a first end, a second end, a thickness, an inner circumference, and an outer circumference, the first end having an opening, and the second end being substantially closed,
wherein the injection molded premade thermoplastic elastomer pre-socket is configured to be:
i) heated to a formable temperature so that injection molded premade thermoplastic elastomer pre-socket becomes formable and stretchable so as to be arranged over at least a portion of a positive model of a residual leg limb and follow contours of an outer surface of the positive model of the residual leg limb, or
ii) heated to a formable temperature so that the injection molded premade thermoplastic elastomer pre-socket becomes formable and stretchable so as to be arranged inside of a rigid prosthetic socket and follow contours of an inner surface of the rigid prosthetic socket, so as to globally reduce an interior volume of the rigid prosthetic socket,
wherein the injection molded premade thermoplastic elastomer pre-socket is configured to be cooled to an ambient temperature after being heated to the formable temperature such that the injection molded premade thermoplastic elastomer pre-socket becomes non-formable, and
wherein the injection molded premade thermoplastic elastomer pre-socket is injected molded in a size to accommodate a specific positive model having a predetermined distal circumference.

21.-40. (canceled)
